SEQUENCE LISTING (95 sequences)

SEQ ID NO: 1 - protein, 36 aa, Leiurus quinquestriatus
  misc_feature: Chlorotoxin
MCMPCFTTDHQMARKCDDCCGGKGRGKCYGPQCLCR

SEQ ID NO: 2 - protein, 42 aa, Leiurus quinquestriatus
HHHHHHMCMPCFTTDHQMARKCDDCCGGKGRGKCYGPQCLCR

SEQ ID NO: 3 - protein, 37 aa, Leiurus quinquestriatus
YMCMPCFTTDHQMARKCDDCCGGKGRGKCYGPQCLCR

SEQ ID NO: 4 - protein, 39 aa, Leiurus quinquestriatus
YSYMCMPCFTTDHQMARKCDDCCGGKGRGKCYGPQCLCR

SEQ ID NO: 5 - protein, 36 aa, Artificial sequence
  Chlorotoxin variant
MCMPCFTTDHQMARKCDDCCGGKGRGKCFGPQCLCR

SEQ ID NO: 6 - protein, 35 aa, Artificial sequence
  Chlorotoxin variant
RCKPCFTTDPQMSKKCADCCGGKGKGKCYGPQCLC

SEQ ID NO: 7 - protein, 38 aa, Artificial sequence
  Chlorotoxin variant
RCSPCFTTDQQMTKKCYDCCGGKGKGKCYGPQCICAPY

SEQ ID NO: 8 - protein, 7 aa, Leiurus quinquestriatus
  misc_feature: Derivative of Chlorotoxin  amino acid residues 23-29
KGRGKSY

SEQ ID NO: 9 - protein, 7 aa, Leiurus quinquestriatus
  misc_feature: Derivative of Chlorotoxin  amino acid residues 8-14
TDHQMAR

SEQ ID NO: 10 - protein, 9 aa, Artificial sequence
  Chlorotoxin alpha peptide
TDHQMARKS

SEQ ID NO: 11 - protein, 9 aa, Artificial sequence
  Variant of chlorotoxin alpha peptide
TAHAMARKS

SEQ ID NO: 12 - protein, 36 aa, Artificial sequence
  Variant peptide of chlorotoxin
MCMPCFTTAHAMARKCDDCCGGKGRCKCYGPQCLCR

SEQ ID NO: 13 - protein, 9 aa, Artificial
  motif for chlorotoxin derivatives
TTXXXMXXK

SEQ ID NO: 14 - protein, 9 aa, Leiurus quinquestriatus
TTDHQMARK

SEQ ID NO: 15 - protein, 35 aa, Mesobuthus tamulus; identical to SEQ ID NO: 6

SEQ ID NO: 16 - protein, 34 aa, Artificial sequence
  Small Toxin consensus sequence
CXPCFTTDXQMAKKCXDCCGGKGKGKCYGPQCLC

SEQ ID NO: 17 - protein, 38 aa, Leiurus quinquestriatus; identical to SEQ ID NO: 7

SEQ ID NO: 18 - protein, 34 aa, Artificial sequence
  Probable Toxin LQH 8/6 consensus sequence
CXPCFTTDXQMXKKCXDCCGGKGKGKCYGPQCIC

SEQ ID NO: 19 - protein, 61 aa, Mesobuthus martensii
  MISC_FEATURE (1)..(61): Xaa can be any amino acid
MKFLYGIVFIALFLTVMFATQTDGCGPCFTTDANMARKCRECCGGIGXXKCFGPQCLCNRI

SEQ ID NO: 20 - protein, 34 aa, Artificial sequence
  Chinese Scorpion consensus sequence
CXPCFTTDXNMARKCXDCCGGXGXXKCFGPQCLC

SEQ ID NO: 21 - protein, 59 aa, Mesobuthus martensii
MKFLYGIVFIALFLTVMFATQTDGCGPCFTTDANMARKCRECCGGIGKCFGPQCLCNRI

SEQ ID NO: 22 - protein, 32 aa, Artificial sequence
  Chinese Scorpion consensus sequence
CXPCFTTDXNMARKCXDCCGGXGKXXXXXXXC

SEQ ID NO: 23 - protein, 37 aa, Mesobuthus eupeus
  MISC_FEATURE (1)..(37): Xaa can be any amino acid
MCMPCFTTDPNMANKCRDCCGGXGKXKCFGPQCLCNR

SEQ ID NO: 24 - protein, 35 aa, Artificial sequence
  Insect toxin I5 consensus sequence
MCMPCFTTDXNMAXKCXDCCGGXGKXKCFGPQCLC

SEQ ID NO: 25 - protein, 35 aa, Mesobuthus eupeus
MCMPCFTTDPNMANKCRDCCGGGKKCFGPQCLCNR

SEQ ID NO: 26 - protein, 33 aa, Artificial sequence
  Insect toxin I5 consensus sequence
MCMPCFTTDXNMAXKCXDCCGGXXKXXXXXXXC

SEQ ID NO: 27 - protein, 38 aa, Mesobuthus eupeus
  MISC_FEATURE (1)..(38): Xaa can be any amino acid
MCMPCFTTRPDMAQQCRACCKGXXRGKCFGPQCLCGYD

SEQ ID NO: 28 - protein, 35 aa, Artificial sequence
  Insectotoxin I1 consensus sequence
MCMPCFTTXXXMAXXCXXCCXGXXRGKCFGPQCLC

SEQ ID NO: 29 - protein, 36 aa, Mesobuthus eupeus
MCMPCFTTRPDMAQQCRACCKGRGKCFGPQCLCGYD

SEQ ID NO: 30 - protein, 33 aa, Artificial sequence
  Insectotoxin I1 consensus sequence
MCMPCFTTXXXMAXXCXXCCXGKGKXXXXXXXC

SEQ ID NO: 31 - protein, 37 aa, Mesobuthus eupeus
  MISC_FEATURE (1)..(37): Xaa can be any amino acid
MCMPCFTTDPNMAKKCRDCCGGNGXXKCFGPQCLCNR

SEQ ID NO: 32 - protein, 35 aa, Artificial sequence
  Insectotoxin 15A consensus sequence
MCMPCFTTDXNMAKKCXDCCGGXGXXKCFGPQCLC

SEQ ID NO: 33 - protein, 35 aa, Mesobuthus eupeus
MCMPCFTTDPNMAKKCRDCCGGNGKCFGPQCLCNR

SEQ ID NO: 34 - protein, 33 aa, Artificial sequence
  Insectotoxin 15A consensus sequence
MCMPCFTTDXNMAKKCXDCCGGXGKXXXXXXXC

SEQ ID NO: 35 - protein, 37 aa, Androctonus mauretanicus
  MISC_FEATURE (1)..(37): Xaa can be any amino acid
CGPCFTTDPYTESKCATCCGGXXRGKCVGPQCLCNRI

SEQ ID NO: 36 - protein, 34 aa, Artificial sequence
  Neurotoxin P2 consensus sequence
CXPCFTTDXXXXXKCXXCCGGXXRGKCXGPQCLC

SEQ ID NO: 37 - protein, 35 aa, Androctonus mauretanicus
CGPCFTTDPYTESKCATCCGGRGKCVGPQCLCNRI

SEQ ID NO: 38 - protein, 32 aa, Artificial sequence
  Neurotoxin P2 consensus sequence
CXPCFTTDXXXXXKCXXCCGGKGKXXXXXXXC

SEQ ID NO: 39 - protein, 37 aa, Artificial sequence
  Toxin consensus sequence
MCMPCFTTDPNMAKKCRDCCGGKGXXKCFGPQCLCNR

SEQ ID NO: 40 - protein, 35 aa, Artificial sequence
  Toxin consensus sequence
RCXPCFTTDXQMSKKCXDCCGGKGKGKCYGPQCLC

SEQ ID NO: 41 - protein, 35 aa, Artificial sequence
  Toxin consensus sequence
MCMPCFTTDPNMARKCRDCCGGRGKCFGPQCLCNR

SEQ ID NO: 42 - protein, 10 aa, Artificial sequence
  Pep8-Ctlx
CGGKGRGKCY

SEQ ID NO: 43 - protein, 10 aa, Artificial sequence
  Pep8-SCX1_BUTSI
CGGKGKGKCY

SEQ ID NO: 44 - protein, 10 aa, Artificial sequence
  Pep8-AF079059_2
CGGIGKCFGP

SEQ ID NO: 45 - protein, 12 aa, Artificial sequence
  Chlorotoxin Peptide 8 consensus sequence
CGGXGRGKCFGP

SEQ ID NO: 46 - protein, 6 aa, Artificial sequence
  Chlorotoxin Peptide 8 consensus sequence
CGGXGK

SEQ ID NO: 47 - protein, 10 aa, Artificial sequence
  Pep8-NJ0361 sequence
CGGGKKCFGP

SEQ ID NO: 48 - protein, 12 aa, Artificial sequence
  Chlorotoxin Peptide 8 consensus sequence
CGGKGKGKCFGP

SEQ ID NO: 49 - protein, 6 aa, Artificial sequence
  Chlorotoxin Peptide 8 consensus sequence
CGGXXK

SEQ ID NO: 50 - protein, 10 aa, Artificial sequence
  Pep8-SCX1_BUTEU sequence
CKGRGKCFGP

SEQ ID NO: 51 - protein, 12 aa, Artificial sequence
  Chlorotoxin Peptide 8 consensus sequence
CGXKGRGKCFGP

SEQ ID NO: 52 - protein, 6 aa, Artificial sequence
  Chlorotoxin Peptide 8 consensus sequence
CXGKGK

SEQ ID NO: 53 - protein, 10 aa, Artificial sequence
  Pep8-SCX5_BUTEU sequence
CGGNGKCFGP

SEQ ID NO: 54 - protein, 12 aa, Artificial sequence
  Chlorotoxin Peptide 8 consensus sequence
CGGXGRGKCFGP

SEQ ID NO: 55 - protein, 6 aa, Artificial sequence
  Chlorotoxin Peptide 8 consensus sequence
CGGXGK

SEQ ID NO: 56 - protein, 10 aa, Artificial sequence
  Pep8-SCXP_ANDMA sequence
CGGRGKCVGP

SEQ ID NO: 57 - protein, 12 aa, Artificial sequence
  Chlorotoxin Peptide 8 consensus sequence
CGGKGRGKCXGP

SEQ ID NO: 58 - protein, 6 aa, Artificial sequence
  Chlorotoxin Peptide 8 consensus sequence
CGGKGK

SEQ ID NO: 59 - protein, 12 aa, Artificial sequence
  Chlorotoxin Peptide 8 consensus sequence
CGGXXRGKCFGP

SEQ ID NO: 60 - protein, 10 aa, Artificial sequence
  Chlorotoxin Peptide 8 consensus sequence
CGGKGKCFGP

SEQ ID NO: 61 - protein, 10 aa, Artificial sequence
  Chlorotoxin Peptide 21 sequence
TTDHQMARKC

SEQ ID NO: 62 - protein, 10 aa, Artificial sequence
  Pep21-SCX1-BUTSI sequence
TTDPQMSKKC

SEQ ID NO: 63 - protein, 10 aa, Artificial sequence
  Chlorotoxin Peptide 21 consensus sequence
TTDXQMAKKC

SEQ ID NO: 64 - protein, 10 aa, Artificial sequence
  Pep21-SCX8_LEIQH sequence
TTDQQMTKKC

SEQ ID NO: 65 - protein, 10 aa, Artificial sequence
  Chlorotoxin Peptide 21 consensus sequence
TTDXQMXKKC

SEQ ID NO: 66 - protein, 10 aa, Artificial sequence
  Pep21-AF079059_2 sequence
TTDANMARKC

SEQ ID NO: 67 - protein, 10 aa, Artificial sequence
  Chlorotoxin Peptide 21 consensus sequence
TTDXNMARKC

SEQ ID NO: 68 - protein, 10 aa, Artificial sequence
  Pep21-JN0361 sequence
TTDPNMANKC

SEQ ID NO: 69 - protein, 10 aa, Artificial sequence
  Chlorotoxin Peptide 21 consensus sequence
TTDXNMAXKC

SEQ ID NO: 70 - protein, 10 aa, Artificial sequence
  Pep21-SCX1_BUTEU sequence
TTRPDMAQQC

SEQ ID NO: 71 - protein, 10 aa, Artificial sequence
  Chlorotoxin Peptide 21 consensus sequence
TTXXXMAXXC

SEQ ID NO: 72 - protein, 10 aa, Artificial sequence
  Pep21-SCX5_BUTEU sequence
TTDPNMAKKC

SEQ ID NO: 73 - protein, 10 aa, Artificial sequence
  Chlorotoxin Peptide 21 consensus sequence
TTDXNMAKKC

SEQ ID NO: 74 - protein, 10 aa, Artificial sequence
  Pep21-SCXP_ANDMA sequence
TTDPYTESKC

SEQ ID NO: 75 - protein, 10 aa, Artificial sequence
  Chlorotoxin Peptide 21 consensus sequence
TTDXXXXXKC

SEQ ID NO: 76 - protein, 10 aa, Artificial sequence
  Chlorotoxin Peptide 21 consensus sequence
TTDPNMAKKC

SEQ ID NO: 77 - protein, 7 aa, Artificial sequence
  Chlorotoxin derivative STP-1
TDPQMSR

SEQ ID NO: 78 - protein, 10 aa, Artificial sequence
  Peptide 8 sequences
GGKGRGKSYG

SEQ ID NO: 79 - protein, 9 aa, Artificial sequence
  Peptide 8a sequence
GKGRGKSYG

SEQ ID NO: 80 - protein, 8 aa, Artificial sequence
  Peptide 8b sequence
KGRGKSYG

SEQ ID NO: 81 - protein, 7 aa, Artificial sequence
  Peptide 8c sequence
GRGKSYG

SEQ ID NO: 82 - protein, 10 aa, Artificial sequence
  Peptide 21 sequence
TTDHQMARKS

SEQ ID NO: 83 - protein, 8 aa, Artificial sequence
  Peptide 21b sequence
DHQMARKS

SEQ ID NO: 84 - protein, 7 aa, Artificial sequence
  Peptide 21c sequence
HQMARKS

SEQ ID NO: 85 - protein, 6 aa, Artificial sequence
  Peptide 21d sequence
QMARKS

SEQ ID NO: 86 - protein, 9 aa, Artificial sequence
  Peptide 21a-A1 sequence
ADHQMARKS

SEQ ID NO: 87 - protein, 9 aa, Artificial sequence
  Peptide 21a-A2 sequence
TAHQMARKS

SEQ ID NO: 88 - protein, 9 aa, Artificial sequence
  Peptide 21a-A3 sequence
TDAQMARKS

SEQ ID NO: 89 - protein, 9 aa, Artificial sequence
  Peptide 21a-A4 sequence
TDHAMARKS

SEQ ID NO: 90 - protein, 9 aa, Artificial sequence
  Peptide 21a-A5 sequence
TDHQAARKS

SEQ ID NO: 91 - protein, 9 aa, Artificial sequence
  Peptide 21a-A7 sequence
TDHQMAAKS

SEQ ID NO: 92 - protein, 9 aa, Artificial sequence
  Peptide 21a-A8 sequence
TDHQMARAS

SEQ ID NO: 93 - protein, 9 aa, Artificial sequence
  Peptide 21a-A9 sequence
TDHQMARKA

SEQ ID NO: 94 - protein, 9 aa, Mesobuthus tamulus sindicus
  MISC_FEATURE (1)..(9): GenBank Accesssion No. P15229, small toxin
TTDQQMSKK

SEQ ID NO: 95 - protein, 9 aa, Leiurus quinquestriatus hebraeu
  Misc_feature (1)..(9): GenBank Accession No. P55966, probable toxin
TTDPQMSKK

(19) United States
(12) Patent Application Publication
(10) Pub. No.: US 2010/0215575 A1
(43) Pub. Date: Aug. 26, 2010

(54) Systemic Administration of Chlorotoxin Agents for the Diagnosis and Treatment of Tumors

(75) Inventors: Alison O'Neill, Boston, MA (US); Douglas B. Jacoby, Wellesley, MA (US)

Correspondence Address: CHOATE, HALL & STEWART LLP, TWO INTERNATIONAL PLACE, BOSTON, MA, 02110, US

(73) Assignee: TRANSMOLECULAR, INC., Cambridge, MA (US)

(21) Appl. No.: 12/682,692

(22) PCT Filed: Oct. 10, 2008
(86) PCT No.: PCT/US08/79547

§ 371 Date: Apr. 12, 2010

Related U.S. Application Data
(60) Provisional application No. 60/979,714, filed on Oct. 12, 2007.

(51) Int. Cl. A61K 51/08; A61K 38/17; A61K 38/43; A61K 49/00; A61P 35/00

(52) U.S. Cl. 424/1.69; 514/12; 424/94.3; 424/9.1; 424/9.6

(57) ABSTRACT

The present invention is directed to methods and compositions for the treatment and diagnosis of neoplastic tumors. The inventive methods generally include systemic (e.g., intravenous) administration of a chloroxotoxin agent that may or may not be labeled.

### RELATED APPLICATION INFORMATION

This application claims benefit of and priority to U.S. Provisional Patent Application 60/979,714 filed Oct. 12, 2007, the contents of which are hereby incorporated by reference in their entirety.

### BACKGROUND

Chlorotoxin, found in the venom of the Giant Yellow Israeli scorpion Leiurus Quinquestriatus, has been shown to exhibit great promise as an agent for the diagnosis and treatment of cancer. Originally described as a chloride-ion channel blocker, the 36-amino acid chlorotoxin peptide has been explored pre-clinically as a candidate for targeting gliomas with 131-iodine (J. A. DeBin et al., Am. J. Physiol. (Cell Physiol.), 1993, 264, 33: C361-C369; L. Soroceanu et al., Cancer Res., 1998, 58: 4871-4879; S. Shen et al., Neuro-Oncol., 2005, 71: 113-119). Compositions (see U.S. Pat. Nos. 5,905,027 and 6,429,187, each of which is hereby incorporated by reference in its entirety) and methods (see U.S. Pat. Nos. 6,028,174 and 6,319,891, each of which is hereby incorporated by reference in its entirety) for diagnosing and treating neuroectodermal tumors (e.g., gliomas and meningiomas) have been developed based on the ability of chlorotoxin to bind to tumor cells of neuroectodermal origin (Soroceanu et al., Cancer Res., 1998, 58: 4871-4879; Ullrich et al., Neuroreport, 1996, 7: 1020-1024; Ullrich et al., Am. J. Physiol., 1996, 270: C1511-C1521).

TM-601, a synthetic version of the naturally-occurring chlorotoxin, has been shown to cross blood brain and tissue barriers. Preclinical studies have demonstrated the stability, safety, efficacy, and lack of immunogenicity of radio-iodinated TM-601. Based on these data, clinical studies (phase I/II) have been performed to evaluate the safety, tolerability, biodistribution and dosimetry of intracavitary delivery of ¹³¹I-TM-601 in adult patients with recurrent high-grade glioma. As of February 2007, out of the 18 patients that have received a single intracavitary dose of ¹³¹I-TM-601 in the Phase I trial, 5 survived 12 months or longer from recurrence; 2 survived more than 36 months from recurrence; and 1 patient remains alive (more than 4 years from recurrence) (see U.S. patent application Ser. No. 11/731,661 and International Application No. PCT/US2007/08309 filed on Mar. 30, 2007, each of which is incorporated herein by reference in its entirety).

The results obtained in these clinical studies are tremendously promising and demonstrate the efficacy of chlorotoxin for the diagnosis and treatment of tumors. In the clinical studies described above, chlorotoxin was administered using an intracavitary route. In the case of tumors located in the brain, intracavitary delivery is initiated during surgery. Thus in cases where surgery is not required or not desirable, intracavitary administration may not be the most appropriate delivery route. Therefore, there is a need for alternative strategies for the administration of chlorotoxin and chlorotoxin-based agents for the diagnosis and treatment of tumors. Particularly desirable are methods of administration that are less invasive than intracavitary delivery.

### SUMMARY OF THE INVENTION

The present invention encompasses the finding that chlorotoxin can be effectively delivered to a subject via systemic administration rather than local administration (e.g., intracavitary). In particular, the present Applicant has demonstrated that chlorotoxin can be effectively delivered intravenously. According to the present invention, systemic delivery to a subject achieves tumor-specific localization of chlorotoxin and results in enhanced survival time.

These and other objects, advantages and features of the present invention will become apparent to those of ordinary skill in the art having read the following detailed description of the preferred embodiments.

### DESCRIPTION OF THE DRAWING

FIG. 1 is a table showing a summary of the binding of TM-601 to various cultured cells (see Example 1 for experimental details).

FIG. 2 is a graph showing the binding of biotinylated TM-601 (TM-602) to multiple cancer cell types using a plate binding assay (see Example 1 for experimental details). Binding is graphed as a percent streptavidin-HRP control relative to cells in which no TM-602 was added. Glioma cells: D54, U251, U373, G26; breast tumor cells: 2LMP, DY3672, LCC6, BT474, SK-BR-3, MCF-7, MDA-MB-231, MDA-MB-468, and MDA-MB-453; non-small cell lung carcinoma cells: A427, WI-62, and H1466; melanoma cells: SKM28; colorectal cancer cells: SW948; and prostate cancer cells: PC3, LNCaP, and DU145.

FIG. 3 is a table showing a summary of the binding of TM-601 to various human tissues.

FIG. 4 illustrates the specific binding of TM-601 to glioblastoma multiforme tumor. Human normal brain and glioblastoma multiforme tumor tissues were histochemically stained with biotinylated TM-601 (left) or buffered saline (right). After primary incubation with biotinylated TM-601 or buffered saline (as a peroxidase reagent staining control), the tissues were incubated with peroxidase-labeled streptavidin followed by the peroxidase substrate to produce brown color in positive samples, which bound the biotinylated TM-601. TM-601 staining is only seen in the tumor tissue (bottom left).

FIG. 5 illustrates the specific binding of TM-601 to human tumor tissues vs. normal tissue. (A) shows representative examples of human tumor tissues histochemically stained with biotinylated TM-601 (A, left) or buffered saline (A, right). (B) shows representative examples of human normal tissues matched to human tumor tissues in (A) histochemically stained with biotinylated TM-601 (B, left) or buffered saline (B, right). After primary incubation with biotinylated TM-601 or buffered saline (as peroxidase reagent staining control), the tissues in (A) and (B) were incubated with peroxidase-labeled streptavidin followed by the peroxidase substrate to produce brown color in samples which bound the biotinylated TM-601. Intense brown color indicative of positive staining was only seen in tumor tissues exposed to biotinylated TM-601 (A, left).

FIG. 6(A) illustrates the efficacy of ¹³¹I-TM-601 in U251-MG brain cancer xenografts in a nude mouse model. Data on the graph are plotted as a Kaplan-Meier Survival Chart. The results obtained showed that the median survival was 29 days for the saline group and 21 days for the cold TM-601 group. In striking contrast, the median survival for the ¹³¹I-TM-601 group was 78 days. FIG. 6(B) shows gamma camera images of two mice (first line: mouse 006; second line: mouse 009) with intracranial xenografts of human U251-MG glioma tumors after injection of ¹³¹I-TM-601. Twenty-one (21) days after the mice had tumor cells implanted, ¹³¹I-TM-601 was injected into the tumor site. Twenty-four (24) and 96 hours after injection, mice were imaged with a gamma camera. Images at 24 and 96 hours showed the excellent retention of radioactivity at the tumor site.

FIG. 7 is a table summarizing the results of brain targeting by ¹²⁵I-TM-601 and ¹²⁵I-EGF after intravenous injection in a mouse model.

FIG. 8 shows Kaplan-Meier survival curves for mice implanted with D54MG xenografts who were untreated or treated with TM-601.

FIG. 9 is a graph showing the effects on TM-601 and Radiation Therapy (RT) on the growth of D54MG flank tumors in mice.

FIG. 10 is a graph showing plasma levels in mice measured after a single dose of TM-601 via intravenous (IV), intraperitoneal (IP), subcutaneous (SC) or oral (OP) administration.

FIG. 11 is a table summarizing the results of GLP toxicology studies conducted with TM-601 in animals.

FIG. 12 is the dosing scheme used in the Phase I imaging and safety study of intravenous ¹³¹I-TM-601 in patients with recurrent or refractory metastatic solid tumors.

FIG. 13 is a table summarizing the tumor-specific uptake of ¹³¹I-TM-601 following intravenous administration in patients with different types of solid tumors.

FIG. 14 shows gamma camera images recorded 3 hours, 24 hours, and 7 days after intravenous injection of ¹³¹I-TM-601 (30 mCi/0.6 mg) to a patient with prostate cancer.

FIG. 15 shows gamma camera images recorded 3 hours, 24 hours, and 48 hours after intravenous injection of ¹³¹I-TM-601 (30 mCi/0.6 mg) to a patient with non-small cell lung cancer.

FIG. 16 shows gamma camera images recorded 3 hours, 24 hours, and 48 hours after intravenous injection of ¹³¹I-TM-601 (30 mCi/0.6 mg) to a patient with malignant glioma.

FIG. 17 shows whole body gamma camera images recorded 24 and 48 hours after intravenous injection of ¹³¹I-TM-601 to a patient with melanoma metastatic to the brain, lung, liver, and a subcutaneous nodule on the right leg.

FIG. 18(A) shows a pre-treatment Magnetic Resonance Image (MRI) showing the left frontal brain metastasis of a patient with metastatic melanoma (left), and a SPECT image recorded 24 hours after intravenous injection of ¹³¹I-TM-601 (30 mCi/0.2 mg) to the patient (right). FIG. 18(B) shows a pre-treatment MRI showing the right occipital brain metastasis of the same patient with metastatic melanoma (left), and a SPECT image recorded 24 hours after intravenous injection of ¹³¹I-TM-601 (10 mCi/0.2 mg) to the patient (right).

FIG. 19 shows a pre-treatment MRI showing left frontal tumor of a patient with malignant glioma (left), and SPECT images taken 48 hours after intravenous injection of ¹³¹I-TM-601 to the patient (right).

FIG. 20 shows a pre-treatment brain MRI of a patient with malignant glioma (left), an SPECT images taken 24 hours after intravenous injection of ¹³¹I-TM-601 (10 mCi/0.2 mg) to the patient (right).

FIG. 21 shows MRIs taken before treatment of a patient with malignant glioma (the same patient as shown in FIG. 20) (left) and 3 weeks after intravenous injection of ¹³¹I-TM-601 to the patient (right).

FIG. 22 shows the half-lives of PEGylated chlorotoxin (TM-601-PEG) as compared to unmodified TM-601 in intravenously injected non-cancerous mice. PEGylation increased the half-life of TM601 by approximately 32-fold.

FIG. 23 shows that PEGylated TM-601 can achieve increased AUC (area under the curve) as shown by increased anti-angiogenic effects with less frequent dosing than unmodified TM-601 in a mouse CNV model. Microvessel density in a CNV model was plotted for various dosing regimens for unmodified TM-601 or for PEGylated TM-601.

### DEFINITIONS

Throughout the specification, several terms are employed that are defined in the following paragraphs.

The terms “approximately” and “about”, as used herein in reference to a number generally includes numbers that fall within a range of 10% in either direction of the number (greater than or less than the number) unless otherwise stated or otherwise evident from the context (except where such number would exceed 100% of a possible value).

The term “biologically active”, when used herein to characterize a polypeptide, refers to a molecule that shares sufficient amino acid sequence homology with a parent polypeptide to exhibit similar or identical properties than the polypeptide (e.g., ability to specifically bind to cancer cells and/or to be internalized into cancer cells and/or to kill cancer cells).

As used herein, the term “cancer” refers to or describes the physiological condition in mammals that is typically characterized by unregulated cell growth. Examples of cancers include, but are not limited to carcinoma, lymphoma, blastoma, sarcoma, and leukemia. More particularly, examples of such cancers include lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

As used herein, the term “cancer cell” refers to a cell in a mammal (e.g., a human being) in vivo which undergoes undesired and unregulated cell growth or abnormal persistence or abnormal invasion of tissues. In vitro, this term also refers to a cell line that is a permanently immortalized established cell culture that will proliferate indefinitely and in an unregulated manner given appropriate fresh medium and space.

As used herein, the term “cancer patient” can refer to an individual suffering from or susceptible to cancer. A cancer patient may or may not have been diagnosed with cancer. The term also includes individuals that have previously undergone therapy for cancer.

The terms “chemotherapeutics” and “anti-cancer agents or drugs” are used herein interchangeably. They refer to those medications that are used to treat cancer or cancerous conditions. Anti-cancer drugs are conventionally classified in one of the following group: alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Examples of such anti-cancer agents include, but are not limited to, BCNU, cisplatin, gemcitabine, hydroxyurea, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomysin, tamoxifen, flutamide, leuprolide, goserelin, aminogluthimide, anastrozole, amsacrine, asparaginase, mitoxantrone, mitotane and amifostine.

The term “cytotoxic”, when used herein to characterize a moiety, compound, drug or agent refers to a moiety, compound, drug or agent that inhibits or prevents the function of cells and/or causes destruction of cells.

As used herein, the term “effective amount” refers to any amount of a compound or composition that is sufficient to fulfill its intended purpose(s), i.e., a desired biological or medicinal response in a tissue or subject. For example, in certain embodiments of the present invention, the purpose(s) may be: to specifically bind to a target tissue, to slow down or stop the progression, aggravation, or deterioration of the symptoms of a cancer, to bring about amelioration of the symptoms of the cancer, and/or to cure the cancer.

The term “fusion protein” refers to a molecule comprising two or more proteins or fragments thereof linked by a covalent bond via their individual peptide backbones, most preferably generated through genetic expression of a polynucleotide molecule encoding those proteins.

The term “homologous” (or “homology”), as used herein, refers to a degree of identity between two polypeptides molecules or between two nucleic acid molecules. When a position in both compared sequences is occupied by the same base or amino acid monomer subunit, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid residues. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, DC, Suppl. 3, 22: 354-352).

The terms “individual” and “subject” are used herein interchangeably. They refer to a human or another mammal (e.g., mouse, rat, rabbit, dog, cat, cattle, swine, sheep, horse or primate) that can be afflicted with or is susceptible to a disease or disorder (e.g., cancer) but may or may not have the disease or disorder. In many embodiments, the subject is a human being. Unless otherwise stated, the terms “individual” and “subject” do not denote a particular age, and thus encompass adults, children, and newborns.

The terms “labeled” and “labeled with a detectable agent or moiety” are used herein interchangeably to specify that an entity (e.g., a chlorotoxin or chlorotoxin conjugate) can be visualized, for example following binding to another entity (e.g., a neoplastic tumor tissue). Preferably the detectable agent or moiety is selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionuclides, fluorophores, chemiluminescent agents, microparticles, enzymes, colorimetric labels, magnetic labels, haptens, Molecular Beacons, aptamer beacons, and the like.

The terms “normal” and “healthy” are used herein interchangeably. They refer to an individual or group of individuals who do not have a tumor. The term “normal” is also used herein to qualify a tissue sample isolated from a healthy individual.

A “pharmaceutical composition” is herein defined herein as a composition that comprises an effective amount of at least one active ingredient (e.g., a chlorotoxin or chlorotoxin conjugate that may or may not be labeled), and at least one pharmaceutically acceptable carrier.

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, 18^{th} Ed., 1990, Mack Publishing Co.: Easton, Pa., which is incorporated herein by reference in its entirety).

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative slicing or limited proteolysis), or in the alternative, may arise from differential post-translational modification (e.g., glycosylation, acylation or phosphorylation).

The term “protein analog”, as used herein, refers to a polypeptide that possesses a similar or identical function as a parent polypeptide but need not necessarily comprise an amino acid sequence that is similar or identical to the amino acid sequence of the parent polypeptide, or possess a structure that is similar or identical to that of the parent polypeptide. Preferably, in the context of the present invention, a protein analog has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the parent polypeptide. Moreover, those of ordinary skill in the art will understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90%, 96%, 97%, 98% or 99% in one or more highly conserved regions usually encompassing at least 3-4 and often up to 20 or more amino acids, with the parent polypeptide, is encompassed in the term “protein analog).

The term “protein fragment”, as used herein, refers to a polypeptide comprising an amino acid sequence of at least 5 amino acid residues of the amino acid sequence of a second polypeptide. A fragment of a protein may or may not possess a functional activity of the parent polypeptide.

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. Small molecules suitable for use in the present invention usually have molecular weight less than about 5,000 daltons (Da), preferably less than about 2,500 Da, more preferably less than 1,000 Da, most preferably less than about 500 Da.

As used herein, the term “systemic administration” refers to administration of an agent such that the agent becomes widely distributed in the body in significant amounts and has a biological effect, e.g., its desired effect, in the blood and/or reaches its desired site of action via the vascular system. Typical systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood.

The terms “therapeutic agent” and “drug” are used herein interchangeably. They refer to a substance, molecule, compound, agent, factor or composition effective in the treatment of a disease or clinical condition.

The term “tissue” is used herein in its broadest sense. A tissue may be any biological entity that can (but does not necessarily) comprise a tumor cell. In the context of the present invention, in vitro, in vivo and ex vivo tissues are considered. Thus, a tissue may be part of an individual or may be obtained from an individual (e.g., by biopsy). Tissues may also include sections of tissue such as frozen sections taken for histological purposes or archival samples with known diagnosis, treatment and/or outcome history. The term tissue also encompasses any material derived by processing the tissue sample. Derived materials include, but are not limited to, cells (or their progeny) isolated from the tissue. Processing of the tissue sample may involve one or more of: filtration, distillation, extraction, concentration, inactivation of interfering components, addition of reagents, and the like.

The term “treatment” is used herein to characterize a method or process that is aimed at (1) delaying or preventing the onset of a disease or condition; (2) slowing down or stopping the progression, aggravation, or deterioration of one or more symptoms of the disease or condition; (3) bringing about ameliorations of the symptoms of the disease or condition; (4) reducing the severity or incidence of the disease or condition; or (5) curing the disease or condition. A treatment may be administered prior to the onset of the disease, for a prophylactic or preventive action. Alternatively or additionally, the treatment may be administered after initiation of the disease or condition, for a therapeutic action.

### DETAILED DESCRIPTION OF CERTAIN PREFERRED EMBODIMENTS

As already mentioned above, the present invention is directed to methods for the treatment and diagnosis of tumors. The methods provided herein generally comprise systemic administration of a chlorotoxin agent that may or may not be labeled with a detectable moiety. In certain preferred embodiments, the chlorotoxin agent is administered intravenously.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual”, 1982; “DNA Cloning: A Practical Approach,” Volumes I and II, D. N. Glover (Ed.), 1985; “Oligonucleotide Synthesis”, M. J. Gait (Ed.), 1984; “Nucleic Acid Hybridization”, B. D. Hames & S. J. Higgins (Eds.), 1985; “Transcription and Translation” B. D. Hames & S. J. Higgins (Eds.),1984; “Animal Cell Culture”, R. I. Freshney (Ed.), 1986; “Immobilized Cells And Enzymes”, IRL Press, 1986; B. Perbal, “A Practical Guide To Molecular Cloning”, 1984.

I. Chlorotoxin Agents

Methods of treatment and diagnostic of the present invention involve systemic administration, to an individual in need thereof, of an effective amount of at least one chlorotoxin agent. As used herein, the term “chlorotoxin agent” refers to a compound that comprises at least one chlorotoxin moiety. In certain embodiments, a chlorotoxin agent comprises at least one chlorotoxin moiety associated with at least one therapeutic moiety (e.g., an anti-cancer agent). The chlorotoxin moiety (and/or therapeutic moiety) may be associated with at least one labeling moiety.

### A. Chlorotoxin Moieties

As used herein, the term “chlorotoxin moiety” refers to a chlorotoxin, a biologically active chlorotoxin subunit or a chlorotoxin derivative.

In certain embodiments, the term “chlorotoxin” refers to the full-length, 36 amino acid polypeptide naturally derived from Leiurus quinquestriatus scorpion venom (DeBin et al., Am. J. Physiol., 1993, 264: C361-369), which comprises the amino acid sequence of native chlorotoxin as set forth in SEQ ID NO. 1 The term “chlorotoxin” includes polypeptides comprising SEQ ID NO. 1 which have been synthetically or recombinantly produced, such as those disclosed in U.S. Pat. No. 6,319,891 (which is incorporated herein by reference in its entirety).

A “biologically active chlorotoxin subunit” is a peptide comprising less than the 36 amino acids of chlorotoxin and which retains at least one property or function of chlorotoxin. As used herein, a “property or function” of chlorotoxin includes, but is not limited to, the ability to arrest abnormal cell growth, ability to specifically bind to a tumor/cancer cell compared to a normal cell, ability to be internalized into a tumor/cancer cell, and/or ability to kill a tumor/cancer cell. The tumor/cancer cell may be in vitro, ex vivo, in vitro, a primary isolate from a subject, a cultured cell, or a cell line.

As used herein, the term “biologically active chlorotoxin derivative” refers to any of a wide variety of derivatives, analogs, variants, polypeptide fragments and mimetics of chlorotoxin and related peptides which retain at least one property or function of chlorotoxin (as described above). Examples of chlorotoxin derivatives include, but are not limited to, peptide variants of chlorotoxin, peptide fragments of chlorotoxin, for example, fragments comprising or consisting of contiguous 10-mer peptides of SEQ ID No. 1, 2, 3, 4, 5, 6, or 7 or comprising residues 10-18 or 21-30 of SEQ ID No. 1, core binding sequences, and peptide mimetics. (See International Application No. PCT/US03/17410, published as WO 2003/101474, the contents of which are hereby incorporated by reference in their entirety.)

Examples of chlorotoxin derivatives include peptides having a fragment of the amino acid sequence set forth in SEQ ID No. 1, having at least about 7, 8, 9, 10, 15, 20, 25, 30 or 35 contiguous amino acid residues, associated with the activity of chlorotoxin. Such fragments may contain functional regions of the chlorotoxin peptide, identified as regions of the amino acid sequence which correspond to known peptide domains, as well as regions of pronounced hydrophilicity. Such fragments may also include two core sequences linked to one another, in any order, with intervening amino acid removed or replaced by a linker.

Derivatives of chlorotoxin include polypeptides comprising a conservative or non-conservative substitution of at least one amino acid residue when the derivative sequence and the chlorotoxin sequence are maximally aligned. The substitution may be one which enhances at least one property or function of chlorotoxin, inhibits at least one property or function of chlorotoxin, or is neutral to at least one property or function of chlorotoxin.

Examples of derivatives of chlorotoxin suitable for use in the practice of the present invention are described in International Application No. WO 2003/101474 (which is incorporated herein by reference in its entirety). Particular examples include polypeptides that comprise or consist of SEQ ID NO. 8 or SEQ ID NO. 13, as well as variants, analogs, and derivatives thereof.

Other examples of chlorotoxin derivatives include those polypeptides containing pre-determined mutations by, e.g., homologous recombination, site-directed or PCR mutagenesis, and the alleles or other naturally-occurring variants of the family of peptides; and derivatives wherein the peptide has been covalently modified by substitution, chemical, enzymatic or other appropriate means with a moiety other than a naturally-occurring amino acid (for example a detectable moiety such as enzyme or a radioisotope).

Chlorotoxin and peptide derivatives thereof can be prepared using any of a wide variety of methods, including standard solid phase (or solution phase) peptide synthesis methods, as is known in the art. In addition, the nucleic acids encoding these peptides may be synthesized using commercially available oligonucleotide synthesis instrumentation and the proteins may be produced recombinantly using standard recombinant production systems.

Other suitable chlorotoxin derivatives include peptide mimetics that mimic the three-dimensional structure of chlorotoxin. Such peptide mimetics may have significant advantages over naturally occurring peptides including, for example, more economical production, greater chemical stability, enhanced pharmacological properties (half-life, absorption, potency, efficacy, etc.), altered specificity (e.g., broad-spectrum biological activities, reduced antigenicity and others).

In certain embodiments, mimetics are molecules that mimic elements of chlorotoxin peptide secondary structure. Peptide backbone of proteins exists mainly to orient amino acid side chains in such a way as to facilitate molecular interactions, such as those of antibody and antigen. A peptide mimetic is expected to permit molecular interactions similar to the natural molecule. Peptide analogs are commonly used in the pharmaceutical industry as non-peptide drugs with properties analogous to those of the template peptide. These types of compounds are also referred to as peptide mimetics or peptidomimetics (see, for example, Fauchere, Adv. Drug Res., 1986, 15: 29-69; Veber & Freidinger, 1985, Trends Neurosci., 1985, 8: 392-396; Evans et al., J. Med. Chem., 1987, 30: 1229-1239) and are usually developed with the aid of computerized molecular modeling.

Generally, peptide mimetics are structurally similar to a paradigm polypeptide (i.e., a polypeptide that has a biochemical property or pharmacological activity), but have one or more peptide linkages optionally replaced by a non-peptide linkage. The use of peptide mimetics can be enhanced through the use of combinatorial chemistry to create drug libraries. The design of peptide mimetics can be aided by identifying amino acid mutations that increase or decrease the binding of a peptide to, for example, a tumor cell. Approaches that can be used include the yeast two hybrid method (see, for example, Chien et al., Proc. Natl. Acad. Sci. USA, 1991, 88: 9578-9582) and using the phase display method. The two hybrid method detects protein-protein interactions in yeast (Field et al., Nature, 1989, 340: 245-246). The phage display method detects the interaction between an immobilized protein and a protein that is expressed on the surface of phages such as lambda and M13 (Amberg et al., Strategies, 1993, 6: 2-4; Hogrefe et al., Gene, 1993, 128: 119-126). These methods allow positive and negative selection of peptide-protein interactions and the identification of the sequences that determine these interactions.

In certain embodiments, a chlorotoxin agent comprises a polypeptide toxin of another scorpion species that displays similar or related activity to chlorotoxin described above. As used herein, the term “similar or related activity to chlorotoxin” refers, in particular, to the selective/specific binding to tumor/cancer cells. Examples of suitable related scorpion toxins include, but are not limited to toxins or related peptides of scorpion origin, that display amino acid and/or nucleotide sequence identity to chlorotoxin. Examples of related scorpion toxins include, but are not limited to, CT neurotoxin from Mesobuthus martenssi (GenBank Accession No. AAD473730), Neurotoxin BmK 41-2 from Buthus martensii karsch (GenBank Accession No. A59356), Neurotoxin Bm12-b from Buthus martensii (GenBank Accession No. AAK16444), Probable Toxin LGH 8/6 from Leiurus quinquestriatus hebraeu (GenBank Accession No. P55966), Small toxin from Mesubutus tamulus sindicus (GenBank Accession No. P15229).

Related scorpion toxins suitable for use in the present invention comprise polypeptides that have an amino acid sequence of at least about 75%, at least about 85%, at least about 90%, at least about 95%, or at least about 99% sequence identity with the entire chlorotoxin sequence as set forth in SEQ ID No. 1. In certain embodiments, related scorpion toxins include those scorpion toxins that have a sequence homologous to SEQ ID NO. 8 or SEQ ID NO. 13.

In certain embodiments, a chlorotoxin moiety within a chlorotoxin agent is labeled. Examples of labeling methods and labeling moieties are described below.

### B. Therapeutic Moieties

As already mentioned above, in certain embodiments, a chlorotoxin agent comprises at least one chlorotoxin moiety associated to at least one therapeutic moiety. Suitable therapeutic moieties include any of a large variety of substances, molecules, compounds, agents or factors that are effective in the treatment of a disease or clinical condition. In certain preferred embodiments, a therapeutic moiety is a chemotherapeutic (i.e., an anti-cancer drug). Suitable anti-cancer drugs include any of a large variety of substances, molecules, compounds, agents or factors that are directly or indirectly toxic or detrimental to cancer cells.

As will be appreciated by one of ordinary skill in the art, a therapeutic moiety may be a synthetic or natural compound: a single molecule, a mixture of different molecules or a complex of different molecules. Suitable therapeutic moieties can belong to any of a variety of classes of compounds including, but not limited to, small molecules, peptides, proteins, saccharides, steroids, antibodies (including fragments and variants thereof), fusion proteins, antisense polynucleotides, ribozymes, small interfering RNAs, peptidomimetics, radionuclides, and the like.

When a therapeutic moiety comprises an anti-cancer drug, the anti-cancer drug can be found, for example, among the following classes of anti-cancer drugs: alkylating agents, anti-metabolic drugs, anti-mitotic antibiotics, alkaloidal anti-tumor agents, hormones and anti-hormones, interferons, non-steroidal anti-inflammatory drugs, and various other anti-tumor agents such as kinase inhibitors, proteasome inhibitors and NF-KB inhibitors.

Examples of anti-cancer drugs include, but are not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.cancer.gov/, a list of the FDA approved oncology drugs at http://wwwfda.gov/cder/cancer/druglistframe.htm, and The Merck Manual, Seventeenth Ed. 1999, the entire contents of which are hereby incorporated by reference.

In certain embodiments, a therapeutic moiety comprises a cytotoxic agent. Examples of cytotoxic agents include toxins, other bioactive proteins, conventional chemotherapeutic agents, enzymes, and radioisotopes.

Examples of suitable cytotoxic toxins include, but are not limited to, bacterial and plant toxins such as gelonin, ricin, saponin, Pseudomonas exotoxin, pokeweed antiviral protein, and diphtheria toxin.

Examples of suitable cytotoxic bioactive proteins include, but are not limited to, proteins of the complement system (or complement proteins). The complement system is a complex biochemical cascade that helps clear pathogens from an organism, and promote healing (B. P. Morgan, Crit. Rev. Clin. Lab. Sci., 1995, 32: 265). The complement system consists of more than 35 soluble and cell-bound proteins, 12 of which are directly involved in the complement pathways.

Examples of suitable cytotoxic chemotherapeutic agents include, but are not limited to, taxanes (e.g., docetaxel, paclitaxel), maytansines, duocarmycins, CC-1065, auristatins, calicheamincins and other enediyne anti-tumor antibiotics. Other examples include the anti-folates (e.g., aminopterin, methotrexate, pemetrexed, raltitrexed), vinca alkaloids (e.g., vincristine, vinblastine, etoposide, vindesine, vinorelbine), and anthracyclines (e.g., daunorubicin, doxorubicin, epirubicin, idarubicin, mitoxantrone, valrubicin).

Examples of suitable cytotoxic enzymes include, but are not limited to, nucleolytic enzymes.

Examples of suitable cytotoxic radioisotopes include any α-, β- or γ-emitter which, when localized at a tumor site, results in cell destruction (S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (Eds.), Academic Press, 1985). Examples of such radioisotopes include, but are not limited to, iodine-131 (¹³¹I), iodine-125 (¹²⁵I), bismuth-212 (²¹²Bi), bismuth-213 (²¹³Bi), astatine-211 (²¹¹At), rhenium-186 (¹⁸⁶Re), rhenium-186 (¹⁸⁸Re), phosphorus-32 (³²P), yttrium-90 (⁹⁰Y), samarium-153 (¹⁵³Sm), and lutetium-177 (¹¹⁷Lu).

Alternatively or additionally, therapeutic moieties suitable for use in the present invention may be any of the therapeutic moieties described in co-owned provisional application entitled “Chlorotoxins as Drug Carriers” (U.S. Ser. No. 60/954,409) filed on Aug. 7, 2007, which is incorporated herein by reference in its entirety. Examples of classes of such therapeutic moieties include, but are not limited to, poorly water soluble anti-cancer agents, anti-cancer agents associated with drug resistance, antisense nucleic acids, ribozymes, triplex agents, short-interfering RNAs (siRNAs), photosensitizers, radiosensitizers, superantigens, prodrug activating enzymes, and anti-angiogenic agents.

### C. Labeling Moieties

In certain embodiments, a chlorotoxin agent is labeled with at least one labeling moiety. For example, one or more chlorotoxin moieties and/or one or more therapeutic moieties within a chlorotoxin agent may be labeled with a labeling moiety.

The role of a labeling moiety is to facilitate detection of the chlorotoxin agent after binding to the tissue to be tested. Preferably, the labeling moiety is selected such that it generates a signal that can be measured and whose intensity is related to (e.g., proportional to) the amount of diagnostic agent bound to the tissue.

Preferably, labeling does not substantially interfere with the desired biological or pharmaceutical activity of the chlorotoxin agent. In certain embodiments, labeling involves attachment or incorporation of one or more labeling moieties to a chlorotoxin moiety, preferably to non-interfering positions on the peptide sequence of the chlorotoxin moiety. Such non-interfering positions are positions that do not participate in the specific binding of the chlorotoxin moiety to tumor cells.

A labeling moiety may be any entity that allows detection of a chlorotoxin agent after binding to a tissue or system of interest. Any of a wide variety of detectable agents can be used as labeling moieties in chlorotoxin agents of the present invention. A labeling moiety may be directly detectable or indirectly detectable. Examples of labeling moieties include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu), fluorescent dyes (for specific exemplary fluorescent dyes, see below), chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper and platinum) or nanoclusters, paramagnetic metal ions, enzymes (for specific examples of enzymes, see below); colorimetric labels (such as, for example, dyes, colloidal gold, and the like), and biotin, digoxigenin, haptens, and proteins for which antisera or monoclonal antibodies are available.

In certain embodiments, a labeling moiety comprises a fluorescent label. Numerous known fluorescent labeling moieties of a wide variety of chemical structures and physical characteristics are suitable for use in the practice of methods of diagnosis of the present invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for coupling fluorescent dyes to other chemical entities such as proteins and peptides, see, for example, “The Handbook of Fluorescent Probes and Research Products”, 9^{th} Ed., Molecular Probes, Inc., Eugene, Oreg. Favorable properties of fluorescent labeling agents include high molar absorption coefficient, high fluorescence quantum yield, and photostability. In certain embodiments, labeling fluorophores desirably exhibit absorption and emission wavelengths in the visible (i.e., between 400 and 750 nm) rather than in the ultraviolet range of the spectrum (i.e., lower than 400 nm).

In certain embodiments, a labeling moiety comprises an enzyme. Examples of suitable enzymes include, but are not limited to, those used in an ELISA, e.g., horseradish peroxidase, beta-galactosidase, luciferase, and alkaline phosphatase. Other examples include beta-glucuronidase, beta-D-glucosidase, urease, glucose oxidase plus peroxide and alkaline phosphatase. An enzyme may be conjugated to a chlorotoxin moiety using a linker group such as a carbodiimide, a diisocyanate, a glutaraldehyde, and the like.

In certain embodiments, a labeling moiety comprises a radioisotope that is detectable by Single Photon Emission Computed Tomography (SPECT) or Position Emission Tomography (PET). Examples of such radionuclides include, but are not limited to, iodine-131 (¹³¹I), iodine-125 (¹²⁵I), bismuth-212 (²¹²Bi), bismuth-213 (²¹³Bi), astatine-221 (²¹¹At), copper-67 (⁶⁷Cu), copper-64 (⁶⁴Cu),) rhenium-186 (¹⁸⁶Re), rhenium-186 (¹⁸⁸Re), phosphorus-32 (³²P), samarium-153 (¹⁵³Sm), lutetium-177 (¹¹⁷Lu), technetium-99m (^{99m}Tc), gallium-67 (⁶⁷Ga), indium-111 (¹¹¹In), and thallium-201 (²⁰¹TI).

In certain embodiments, a labeling moiety comprises a radioisotope that is detectable by Gamma camera. Examples of such radioisotopes include, but are not limited to, iodine-131 (¹³¹I), and technetium-99m (^{99m}Tc).

In certain embodiments, a labeling moiety comprises a paramagnetic metal ion that is a good contrast enhancer in Magnetic Resonance Imaging (MRI). Examples of such paramagnetic metal ions include, but are not limited to, gadolinium III (Gd³⁺), chromium III (Cr^{3|}), dysprosium III (Dy^{3|}), iron III (Fe^{3|}), manganese II (Mn^{2|}), and ytterbium III (Yb^{3|}). In certain preferred embodiments, the labeling moieties comprises gadolinium III (Gd³⁺). Gadolinium is an FDA-approved contrast agent for MRI, which accumulates in abnormal tissues causing these abnormal areas to become very bright (enhanced) on the magnetic resonance image. Gadolinium is known to provide great contrast between normal and abnormal tissues in different areas of the body, in particular in the brain.

In certain embodiments, a labeling moiety comprises a stable paramagnetic isotope detectable by nuclear magnetic resonance spectroscopy (MRS). Examples of suitable stable paramagnetic isotopes include, but are not limited to, carbon-13 (¹³C) and fluorine-19 (¹⁹F).

### D. Formation of Chlorotoxin Agents

In certain embodiments, a chlorotoxin agent comprises at least one chlorotoxin moiety associated with at least one therapeutic moiety. Thus, a chlorotoxin agent results from the association (e.g., binding, interaction, fusion, or coupling) of at least two other molecules.

Association between a chlorotoxin moiety and a therapeutic moiety within a chlorotoxin agent may be covalent or non-covalent. Irrespective of the nature of the binding, interaction, or coupling, the association between a chlorotoxin moiety and a therapeutic moiety is preferably selective, specific and strong enough so that the chlorotoxin agent does not dissociate before or during transport/delivery to and into the tumor. Association between a chlorotoxin moiety and a therapeutic moiety within a chlorotoxin agent may be achieved using any chemical, biochemical, enzymatic, or genetic coupling known to one skilled in the art.

In certain embodiments, association between a chlorotoxin moiety and a therapeutic moiety is non-covalent. Examples of non-covalent interactions include, but are not limited to, hydrophobic interactions, electrostatic interactions, dipole interactions, van der Waals interactions, and hydrogen bonding.

In certain embodiments, association between a chlorotoxin moiety and a therapeutic moiety is covalent. As will be appreciated by one skilled in the art, the moieties may be attached to each other either directly or indirectly (e.g., through a linker, as described below).

In certain embodiments, the chlorotoxin moiety and therapeutic moiety are directly covalently linked to each other. Direct covalent binding can be through a linkage such as an amide, ester, carbon-carbon, disulfide, carbamate, ether, thioether, urea, amine, or carbonate linkage. The covalent binding can be achieved by taking advantage of functional groups present on the chlorotoxin moiety and/or the therapeutic moiety. Alternatively, a non-critical amino acid may be replaced by another amino acid which will introduce a useful group (amino, carboxy or sulfhydryl) for coupling purposes. Alternatively, an additional amino acid may be added to the chlorotoxin moiety to introduce a useful group (amino, carboxy or sulfhydryl) for coupling purposes. Suitable functional groups that can be used to attach moieties together include, but are not limited to, amines, anhydrides, hydroxyl groups, carboxy groups, thiols, and the like. An activating agent, such as a carbodiimide, can be used to form a direct linkage. A wide variety of activating agents are known in the art and are suitable for linking a therapeutic agent and a chlorotoxin moiety.

In other embodiments, a chlorotoxin moiety and a therapeutic moiety within a chlorotoxin agent are indirectly covalently linked to each other via a linker group. This can be accomplished by using any number of stable bifunctional agents well known in the art, including homofunctional and heterofunctional agents (for examples of such agents, see, e.g., Pierce Catalog and Handbook). The use of a bifunctional linker differs from the use of an activating agent in that the former results in a linking moiety being present in the resulting chlorotoxin agent, whereas the latter results in a direct coupling between the two moieties involved in the reaction. The role of a bifunctional linker may be to allow reaction between two otherwise inert moieties. Alternatively or additionally, the bifunctional linker, which becomes part of the reaction product may be selected such that it confers some degree of conformational flexibility to the chlorotoxin agent (e.g., the bifunctional linker comprises a straight alkyl chain containing several atoms, for example, the straight alkyl chain contains between 2 and 10 carbon atoms). Alternatively or additionally, the bifunctional linker may be selected such that the linkage formed between a chlorotoxin moiety and therapeutic moiety is cleavable, e.g. hydrolysable (for examples of such linkers, see e.g. U.S. Pat. Nos. 5,773,001; 5,739,116 and 5,877,296, each of which is incorporated herein by reference in its entirety). Such linkers are for example preferably used when higher activity of the chlorotoxin moiety and/or of the therapeutic moiety is observed after hydrolysis of the conjugate. Exemplary mechanisms by which a therapeutic moiety may be cleaved from a chlorotoxin moiety include hydrolysis in the acidic pH of the lysosomes (hydrazones, acetals, and cis-aconitate-like amides), peptide cleavage by lysosomal enzymes (the capthepsins and other lysosomal enzymes), and reduction of disulfides). Another mechanism by which a therapeutic moiety is cleaved from the chlorotoxin agent includes hydrolysis at physiological pH extra- or intra-cellularly. This mechanism applies when the crosslinker used to couple the therapeutic moiety to the chlorotoxin moiety is a biodegradable/bioerodible entity, such as polydextran and the like.

For example, hydrazone-containing chlorotoxin agents can be made with introduced carbonyl groups that provide the desired release properties. Chlorotoxin agents can also be made with a linker that comprise an alkyl chain with a disulfide group at one end and a hydrazine derivative at the other end. Linkers containing functional groups other than hydrazones also have the potential to be cleaved in the acidic milieu of lysosomes. For example, chlorotoxin agents can be made from thiol-reactive linkers that contain a group other than a hydrazone that is cleavable intracellularly, such as esters, amides, and acetals/ketals.

Another example of class of pH sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used.

Another potential release method for chlorotoxin agents is the enzymatic hydrolysis of peptides by the lysosomal enzymes. In one example, a peptidic toxin is attached via an amide bond to para-aminobenzyl alcohol and then a carbamate or carbonate is made between the benzyl alcohol and the therapeutic moiety. Cleavage of the peptide leads to collapse of the amino benzyl carbamate or carbonate, and release of the therapeutic moiety. In another example, a phenol can be cleaved by collapse of the linker instead of the carbamate. In another variation, disulfide reduction is used to initiate the collapse of a para-mercaptobenzyl carbamate or carbonate.

In embodiments where a therapeutic moiety within a chlorotoxin agent is a protein, a polypeptide or a peptide, the chlorotoxin agent may be a fusion protein. As already defined above, a fusion protein is a molecule comprising two or more proteins or peptides linked by a covalent bond via their individual peptide backbones. Fusion proteins used in methods of the present invention can be produced by any suitable method known in the art. For example, they can be produced by direct protein synthetic methods using a polypeptide synthesizer. Alternatively, PCR amplification of gene fragments can be carried out using anchor primers which give rise to complementary overhangs between two consecutive gene fragments that can subsequently be annealed and re-amplified to generate a chimeric gene sequence. Fusion proteins can be obtained by standard recombinant methods (see, for example, Maniatis et al. “Molecular Cloning: A Laboratory Manual”, 2^{nd} Ed., 1989, Cold Spring Harbor Laboratory, Cold Spring, N.Y.). These methods generally comprise (1) construction of a nucleic acid molecule that encodes the desired fusion protein; (2) insertion of the nucleic acid molecule into a recombinant expression vector; (3) transformation of a suitable host cell with the expression vector; and (4) expression of the fusion protein in the host cell. Fusion proteins produced by such methods may be recovered and isolated, either directly from the culture medium or by lysis of the cells, as known in the art. Many methods for purifying proteins produced by transformed host cells are well-known in the art. These include, but are not limited to, precipitation, centrifugation, gel filtration, and (ion-exchange, reverse-phase, and affinity) column chromatography. Other purification methods have been described (see, for example, Deutscher et al. “Guide to Protein Purification” in Methods in Enzymology, 1990, Vol. 182, Academic Press).

As can readily be appreciated by one skilled in the art, a chlorotoxin agent used in methods of the present invention can comprise any number of chlorotoxin moieties and any number of therapeutic moieties, associated to one another by any number of different ways. The design of a conjugate will be influenced by its intended purpose(s) and the properties that are desirable in the particular context of its use. Selection of a method to associate or bind a chlorotoxin moiety to a therapeutic moiety to form a chlorotoxin agent is within the knowledge of one skilled in the art and will generally depend on the nature of the interaction desired between the moieties (i.e., covalent vs. non-covalent and/or cleavable vs. non-cleavable), the nature of the therapeutic moiety, the presence and nature of functional chemical groups on the moieties involved and the like.

In labeled chlorotoxin agents, association between a chlorotoxin moiety (or therapeutic moiety) and a labeling moiety may be covalent or non-covalent. In case of covalent association, the chlorotoxin (or therapeutic) and labeling moieties may be attached to each other either directly or indirectly, as described above.

In certain embodiments, association between a chlorotoxin moiety (or therapeutic moiety) and a labeling moiety is non-covalent. Examples of non-covalent associations include, but are not limited to, hydrophobic interactions, electrostatic interactions, dipole interactions, van der Waals interactions, and hydrogen bonding. For example, a labeling moiety can be non-covalently attached to a chlorotoxin moiety (or therapeutic moiety) by chelation (e.g., a metal isotope can be chelated to a polyHis region attached, e.g., fused, to a chlorotoxin moiety).

In certain embodiments, a chlorotoxin moiety (or therapeutic moiety) is isotopically labeled (i.e., it contains one or more atoms that have been replaced by an atom having an atomic mass or mass number different from the atomic mass or mass number usually found in nature). Alternatively or additionally, an isotope may be attached to a chlorotoxin moiety and/or therapeutic moiety.

As can readily be appreciated by one skilled in the art, a labeled chlorotoxin agent used in certain methods of the present invention can comprise any number of chlorotoxin moieties, any number of therapeutic moieties, and any number of labeling moieties, associated to one another by any number of different ways. The design of a labeled chlorotoxin agent will be influenced by its intended purpose(s), the properties that are desirable in the context of its use, and the method selected from the detection.

### II. Methods of Treatment

Methods of treatment of the invention include systemic (e.g., intravenous) administration of an effective amount of a chlorotoxin agent, or a pharmaceutical composition thereof, to an individual in need thereof (i.e., a individual with a neoplastic tumor). Thus, methods of treatment of the present invention may be used for reducing the size of solid tumors, inhibiting tumor growth and/or metastasis, treating lymphatic cancers, and/or prolonging the survival time of mammals (including humans) suffering from cancers and cancer conditions.

### A. Indications

Examples of cancers and cancer conditions that can be treated according to the present invention include, but are not limited to, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, haematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., oesophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye and adnexa, thyroid, adrenal gland, and other endocrine glands and related structures, secondary and unspecified malignant neoplasms of lymph nodes, secondary malignant neoplasm of respiratory and digestive systems and secondary malignant neoplasms of other sites.

In certain embodiments of the present invention, inventive compositions and methods are used in the treatment of sarcomas. In some embodiments, compositions and methods of the present invention are used in the treatment of bladder cancer, breast cancer, chronic lymphoma leukemia, head and neck cancer, endometrial cancer, Non-Hodgkin's lymphoma, non-small cell lung cancer, ovarian cancer, pancreatic cancer, and prostate cancer.

In certain embodiments of the present invention, compositions and methods are used for the treatment of tumors of neuroectodermal origin. Any tumor of neuroectodermal origin present in a human patient can generally be treated using a composition/method of the present invention. In certain embodiments, the tumor of neuroectodermal origin affecting the patient is a member of the group consisting of gliomas, meningiomas, ependymomas, medulloblastomas, neuroblastomas, gangliomas, pheochromocytomas, melanomas, peripheral primitive neuroectodermal tumors, small cell carcinoma of the lung, Ewing's sarcoma, and metastatic tumors of neuroectodermal origin in the brain.

In certain embodiments, the tumor of neuroectodermal origin affects the brain of the patient. In certain embodiments, the brain tumor is a glioma. About half of all primary brain tumors are gliomas. There are 4 main types of glioma: astrocytoma (which is the most common type of glioma in both adults and children), ependymoma, oligodendroglioma, and mixed glioma. Gliomas can be classified according to their location: infratentorial (i.e., located in the lower part of the brain, found mostly in children patients) or supratentorial (i.e., located in the upper part of the brain, found mostly in adult patients).

Gliomas are further categorized according to their grade, which is determined by pathologic evaluation of the tumor. The World Health Organization (WHO) has developed a grading system, from Grade I gliomas, which tend to be the least aggressive, to Grade IV gliomas, which tend to be the most aggressive and malignant. Examples of low grade (i.e., Grade I or Grade II) gliomas include, but are not limited to, pilocytic astrocytoma (also called juvenile pilocytic astrocytoma), fibrillary astrocytoma, pleomorphic xantroastrocytomoa, and desembryoplastic neuroepithelial tumor. High-grade gliomas encompass Grade III gliomas (e.g., anaplastic astrocytoma, AA) and Grade IV gliomas (glioblastoma multiforme, GBM). Anaplastic astrocytoma is most frequent among men and women in theirs 30s-50s, and accounts for 4% of all brain tumors. Glioblastoma multiforme, the most invasive type of glial tumor, is most common in men and women in their 50s-70s and accounts for 23% of all primary brain tumors. The prognosis is the worst for Grade IV gliomas, with an average survival time of 12 months. In certain embodiments, methods of the present invention are used for the treatment of high-grade gliomas.

Despite aggressive treatment, gliomas usually recur, often with a higher grade and sometimes with a different morphology. While recurrence varies, Grade IV gliomas invariably recur. Thus, in certain embodiments, methods of the present invention are used for the treatment of recurrent gliomas, in particular, recurrent high-grade gliomas.

Tumors that can be treated using compositions and methods of the present invention also include tumors that are refractory to treatment with other chemotherapeutics. The term “refractory”, when used herein in reference to a tumor means that the tumor (and/or metastases thereof), upon treatment with at least one chemotherapeutic other than an inventive composition, shows no or only weak anti-proliferative response (i.e., no or only weak inhibition of tumor growth) after the treatment with such a chemotherapeutic agent—that is, a tumor that cannot be treated at all or only with unsatisfying results with other (preferably standard) chemotherapeutics. The present invention, where treatment of refractory tumors and the like is mentioned, is to be understood to encompass not only (i) tumors where one or more chemotherapeutics have already failed during treatment of a patient, but also (ii) tumors that can be shown to be refractory by other means, e.g., biopsy and culture in the presence of chemotherapeutics.

Patients that can receive a treatment according to the present invention generally include any patient diagnosed with a neoplastic tumor. As will be recognized by one skilled in the art, different methods of diagnosis may be performed depending on the location and nature of the tumor, including imaging, biopsy, etc.

### B. Dosages and Administrations

In a method of treatment of the present invention, a chlorotoxin agent, or a pharmaceutical composition thereof, will generally be administered in such amounts and for such a time as is necessary or sufficient to achieve at least one desired result. For example, a chlorotoxin agent can be administered in such amounts and for such a time that it kills cancer cells, reduces tumor size, inhibits or delay tumor growth or metastasis, prolongs the survival time of patients, or otherwise yields clinical benefits.

A treatment according to the present invention may consist of a single dose or a plurality of doses over a period of time. Administration may be one or multiple times daily, weekly (or at some other multiple day interval) or on an intermittent schedule. The exact amount of a chlorotoxin agent, or pharmaceutical composition thereof, to be administered will vary from subject to subject and will depend on several factors (see below).

Chlorotoxin agents, or pharmaceutical compositions thereof, may be administered using any systemic administration route effective for achieving the desired therapeutic effect. Typical systemic routes of administration include, but are not limited to, intramuscular, intravenous, pulmonary, and oral routes. Administration may also be performed, for example, by infusion or bolus injection, or by absorption through epithelial or mucocutaneous linings (e.g., oral, mucosa, rectal and intestinal mucosa, etc.). In certain preferred embodiments, the chlorotoxin agent is administered intravenously. Exemplary procedures for the intravenous administration of a chlorotoxin agent in human patients are described in Example 9.

Depending on the route of administration, effective doses may be calculated according to the body weight, body surface area, or organ/tumor size of the subject to be treated. Optimization of the appropriate dosages can readily be made by one skilled in the art in light of pharmacokinetic data observed in human clinical trials. The final dosage regimen will be determined by the attending physician, considering various factors which modify the action of the drugs, e.g., the drug's specific activity, the severity of the damage and the responsiveness of the patient, the age, condition, body weight, sex and diet of the patient, the severity of any present infection, time of administration, the use (or not) of other therapies, and other clinical factors. As studies are conducted using chlorotoxin agents, further information will emerge regarding the appropriate dosage levels and duration of treatment.

Typical dosages comprise 1.0 pg/kg body weight to 100 mg/kg body weight. For example, for systemic administration, dosages may be 100.0 ng/kg body weight to 10.0 mg/kg body weight.

More specifically, in certain embodiments where a chlorotoxin agent is administered intravenously, dosing of the agent may comprise administration of one or more doses comprising about 0.001 mg/kg to about 5 mg/kg, e.g., from about 0.001 mg/kg to about 5 mg/kg, from about 0.01 mg/kg to about 4 mg/kg, from about 0.02 mg/kg to about 3 mg/kg, from about 0.03 mg/kg to about 2 mg/kg or from about 0.03 mg/kg to about 1.5 mg/kg of chlorotoxin. For example, in certain embodiments, one or more doses of chlorotoxin agent may be administered that each contains about 0.03 mg/kg, about 0.04 mg/kg, about 0.05 mg/kg, about 0.06 mg/kg, about 0.07 mg/kg, about 0.09 mg/kg, about 1.0 mg/kg or more than 1.0 mg/kg of chlorotoxin. In other embodiments, one or more doses of chlorotoxin agent may be administered that each contains about 0.05 mg/kg, about 0.10 mg/kg, about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1.0 mg/kg, or more than about 1 mg/kg of chlorotoxin. In yet other embodiments, one or more doses of chlorotoxin agent may be administered that each contains about 1.0 mg/kg, about 1.05 mg/kg, about 1.10 mg/kg, about 1.15 mg/kg, about 1.20 mg/kg, about 1.25 mg/kg, about 1.3 mg/kg, about 1.35 mg/kg, about 1.40 mg/kg, about 1.45 mg/kg, about 1.50 mg/kg, or more than about 1.50 mg/kg of chlorotoxin. In such embodiments, at treatment may comprise administration of a single dose of chlorotoxin agent or administration of 2 doses, 3 doses, 4 doses, 5 doses, 6 doses or more than 6 doses. Two consecutive doses may be administered at 1 day interval, 2 days interval, 3 days interval, 4 days interval, 5 days interval, 6 days interval, 7 days interval, or more than 7 days interval (e.g., 10 days, 2 weeks, or more than 2 weeks).

### C. Combination Therapies

It will be appreciated that methods of treatment of the present invention can be employed in combination with additional therapies (i.e., a treatment according to the present invention can be administered concurrently with, prior to, or subsequently to one or more desired therapeutics or medical procedures). The particular combination of therapies (therapeutics or procedures) to employ in such a combination regimen will take into account compatibility of the desired therapeutics and/or procedures and the desired therapeutic effect to be achieved.

For example, methods of treatment of the present invention can be employed together with other procedures including surgery, radiotherapy (e.g., y-radiation, neutron beam radiotherapy, electron beam radiotherapy, proton therapy, brachytherapy, systemic radioactive isotopes), endocrine therapy, hyperthermia, and cryotherapy, depending on the tumor to be treated.

In many cases of brain tumor, a treatment of the present invention will very often be administered after surgery. In the treatment of brain tumor, the main goal of surgery is to achieve a gross-total resection, i.e., removal of all visible tumor. One of the difficulties in achieving such a goal is that these tumors are infiltrative, i.e., they tend to weave in and out among normal brain structures. Furthermore, there is a great variability in the amount of tumor that can be safely removed from the brain of a patient. Removal is generally not possible if all or part of the tumor is located in a region of the brain controlling critical functions.

In many cases of brain tumor, a treatment of the present invention will often be administered in combination with (i.e., concurrently with, prior to, or subsequently to) radiotherapy. In conventional treatments, radiotherapy generally follows surgery. Radiation is generally given as a series of daily treatments (called fractions) over several weeks. This “fractionated” approach to administering radiation is important to maximize the destruction of tumor cells and minimize side effects on normal adjacent brain. The area over which the radiation is administered (called the radiation field) is carefully calculated to avoid including as much of normal brain as is feasible.

Alternatively or additionally, methods of treatment of the present invention can be administered in combination with other therapeutic agents, such as agents that attenuate any adverse effects (e.g., antiemetics) and/or with other approved chemotherapeutic drugs. Examples of chemotherapeutics include, but are not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Methods of the present invention can also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); Ch1VPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

As will be appreciated by one skilled in the art, the selection of one or more therapeutic agents to be administered in combination with a method of treatment of the present invention will depend on the tumor to be treated.

For example, chemotherapeutic drugs prescribed for brain tumors include, but are not limited to, temozolomide (Temodar®), procarbazine (Matulane®), and lomustine (CCNU), which are taken orally; vincristine (Oncovin® or Vincasar PFS®), cisplatin (Platinol®), carmustine (BCNU, BiCNU), and carboplatin (Paraplatin®), which are administered intravenously; and methotrexate (Rheumatrex® or Trexall®), which can be administered orally, intravenously or intrathecally (i.e., injected directly into spinal fluid). BCNU is also given under the form of a polymer wafer implant during surgery (Giadel® wafers). One of the most commonly prescribed combination therapy for brain tumors is PCV (procarbazine, CCNU, and vincristine) which is usually given every six weeks.

In embodiments where the tumor to be treated is a brain tumor of neuroectodermal origin, a method of the present invention may be used in combination with agents for the management of symptoms such as seizures and cerebral edema. Examples of anticonvulsants successfully administered to control seizures associated with brain tumors include, but are not limited to, phenytoin (Dilantin®), Carbamazepine (Tegretol®) and divalproex sodium (Depakote®). Swelling of the brain may be treated with steroids (e.g., dexamethason (Decadron®).

### D. Pharmaceutical Compositions

As mentioned above, methods of treatment of the present invention include administration of a chlorotoxin agent per se or in the form of a pharmaceutical composition. A pharmaceutical composition will generally comprise an effective amount of at least one chlorotoxin agent and at least one pharmaceutically acceptable carrier or excipient.

Pharmaceutical compositions may be formulated using conventional methods well-known in the art. The optimal pharmaceutical formulation can be varied depending upon the route of administration and desired dosage. Such formulations may influence the physical state, stability, rate of in vivo release, and rate of in vivo clearance of the administered compounds. Formulation may produce solid, liquid or semi-liquid pharmaceutical compositions.

Pharmaceutical compositions may be formulated in dosage unit form for ease of administration and uniformity of dosage. The expression “unit dosage form”, as used herein, refers to a physically discrete unit of chlorotoxin agent for the patient to be treated. Each unit contains a predetermined quantity of active material calculated to produce the desired therapeutic effect. It will be understood, however, that the total dosage of the composition will be decided by the attending physician within the scope of sound medical judgment.

As mentioned above, in certain embodiments, the chlorotoxin agent is administered intravenously through injection or infusion. Pharmaceutical compositions suitable for administration by injection or infusion may be formulated according to the known art using suitable dispersing or wetting agents, and suspending agents. The pharmaceutical composition may also be a sterile injectable solution, suspension or emulsion in a non-toxic diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspension medium. For this purpose, any bland fixed oil can be used including synthetic mono- or di-glycerides. Fatty acids such as oleic acid may also be used in the preparation of injectable formulations.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from injection. This may be accomplished by dissolving or suspending the active ingredient in an oil vehicle. Injectable depot forms are made by forming micro-encapsulated matrices of the drug in biodegradable polymers such as polylactide-polyglycolide. Depending on the ratio of drug to polymer and the nature of the particular polymer employed, the rate of drug release can be controlled. Depot injectable formulations can also be prepared by entrapping the drug in liposomes or microemulsions which are compatible with body tissues.

### III. Methods of Diagnosis

### A. Administration

In another aspect, the present invention provides methods for the in vivo diagnosis of tumors. More specifically, methods are provided for differentiating neoplastic tumor tissue from non-neoplastic tissue in a patient. Such methods include systemically (e.g., intravenously) administering to a patient an effective amount of a labeled chlorotoxin agent described herein, or a pharmaceutical composition thereof, such that specific binding of the labeled chlorotoxin agent to a tissue within the patient can occur, if the tissue is neoplastic.

Generally, the dosage of a labeled chlorotoxin agent will vary depending on considerations such as age, sex, and weight of the patient, area(s) of the body to be examined, as well as the administration route. Factors such as contraindications, concomitant therapies, and other variables are also to be taken into account to adjust the dosage of the labeled chlorotoxin agent to be administered. This can, however, be readily achieved by a trained physician. In general, a suitable dose of a labeled chlorotoxin agent corresponds to the lowest amount of agent that is sufficient to allow detection of neoplastic tumor tissue in the patient.

For example, in embodiments where the chlorotoxin agent is labeled with ¹³¹I and administered intravenously, dosing of the labeled chlorotoxin agent may comprise administration of one or more doses each comprising about 5 mCi to about 100 mCi, e.g., about 5 mCi to about 80 mCi, about 10 mCi to about 80 mCi, or about 10 mCi to about 50 mCi ¹³¹I. For example, one or more doses of ¹³¹I-radiolabeled chlorotoxin agent may be administered that each contains about 10 mCi, about 20 mCi, about 30 mCi ¹³¹I, about 40 mCi ¹³¹I, about 50 mCi ¹³¹I, about 60 mCi ¹³¹I, about 70 mCi ¹³¹I, about 80 mCi ¹³¹I, about 90 mCi ¹³¹I, or about 100 mCi ¹³¹I. In such embodiments, a diagnosis procedure may comprise administration of a single dose of ¹³¹I-radiolabeled chlorotoxin agent or administration of multiple doses, e.g., 2 doses, 3 doses or 4 doses. Two consecutive doses may be administered at 1 day interval, 2 days interval, 3 days interval, 4 days interval, 5 days interval, 6 days interval, 7 days interval or more than 7 days interval.

In embodiments where a ¹³¹I-radiolabeled chlorotoxin agent is used, the patient is preferably administered supersaturated potassium iodide prior to administration of the ¹³¹I-radiolabeled chlorotoxin (e.g., 1 day, 2 days, or 3 days before treatment according to the present invention). Administration of supersaturated potassium iodide blocks uptake of ¹³¹I by the thyroid gland, thus preventing side effects such as hypothyroidism.

Following administration of the labeled chlorotoxin agent and after sufficient time has elapsed for specific binding to take place, detection of the bound labeled chlorotoxin agent is performed.

### B. Tumor Detection and Localization

As will be recognized by one skilled in the art, detection of binding of a labeled chlorotoxin agent to a tissue of interest may be carried out by any of a wide variety of methods including, but not limited to, spectroscopic, photochemical, biochemical, immunochemical, electrical, optical or chemical means. Selection of a detection method will generally be based on the nature of the labeling moiety of the agent (i.e., fluorescent moiety, radionuclide, paramagnetic metal ion, and the like). In certain preferred embodiments, detection and localization of a tumor within a patient are carried out using an imaging technique.

Different imaging techniques can be used depending on the nature of the labeling moiety. For example, the binding may be detected using Magnetic Resonance Imaging (MRI) if the labeling moiety comprises a paramagnetic metal ion (e.g., Gd³⁺). Single Photon Emission Computed Tomography (SPECT) and/or Positron Emission Tomography (PET) can be used for binding detection if the labeling moiety comprises a radioisotope (e.g., ¹³¹I, and the like). Other imaging techniques include gamma camera imaging.

### C. Diagnosis

According to diagnostic methods of the present invention, a tissue is identified as a neoplastic tissue if the level of binding of the labeled chlorotoxin agent to the tissue of interest is elevated compared to the level of binding of the labeled chlorotoxin agent to a normal tissue. As already mentioned above, a normal tissue is herein defined as a non-neoplastic tissue. For example, when the method is performed in vivo, the level of binding of the labeled chlorotoxin agent measured in a region of an organ of interest (e.g., the brain) may be compared to the level of binding of the labeled chlorotoxin agent measured in a normal region of the same organ.

In certain embodiments, the tissue of interest is identified as a neoplastic tissue if the level of binding measured is higher than the level of binding to a normal tissue. For example, the level of binding may be at least about 2 times higher, at least about 3 times higher, at least about 4 times higher, at least about 5 times higher, at least about 10 times higher, at least about 25 times higher, at least about 50 times higher, at least about 75 times higher, at least about 100 times higher, at least about 150 times higher, at least about 200 times higher, or more than 200 times higher than the level of binding to a normal tissue.

### Examples

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### In Vitro Cell Binding of TM-601 and Biotinylated TM-601 (TM-602)

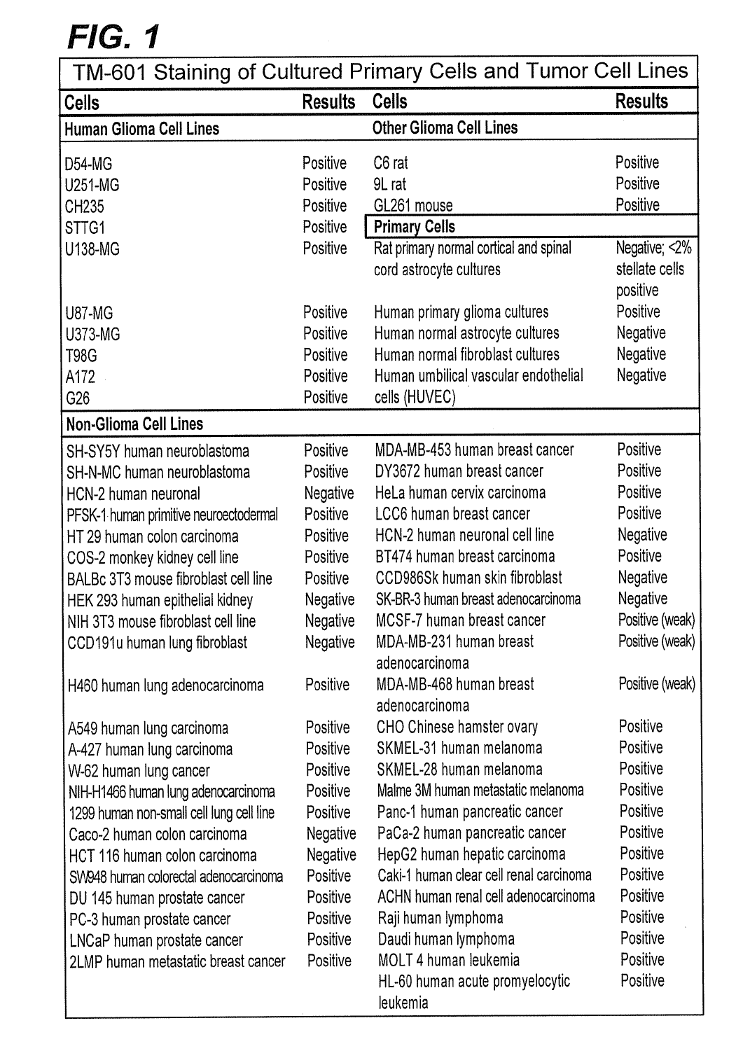

The binding of TM-601, a 36 amino acid peptide originally isolated from Leiurus quinquestriatus scorpion venom, to tumor cell lines and normal primary cell cultures has been studied. Results obtained further corroborated the previous preliminary observation that TM-601 binds selectively to many different tumor types. Human, rat, and mouse glioma cell lines, cultured primary cells, and non-glioma cell lines, including those from human, monkey, rat, hamster, and mouse, were tested for TM-601 binding. Additionally, many non-glioma tumor lines including for example tumor lines derived from lung, colon, prostate and melanoma tumors were also found to bind TM-601. In contrast, under the experimental conditions used, a number of primary cultured cells (rat and human astrocytes) and non-glioma cell lines (human lung fibroblast, human skin fibroblast, human umbilical vascular endothelial cells, human neuronal cells, and 3T3 mouse fibroblasts) were found to be negative for TM-601 binding. All the data obtained are summarized in FIG. 1.

Demonstration of TM-601 Binding on Cells in Plate Binding and by FACS assays: Biotinylated TM-601 (TM-602) was used in a plate binding assay to demonstrate targeting of a broad variety of tumor cell lines representing various cancer types. Human cancer cell lines tested included metastatic and primary breast, lung, prostate, brain, colorectal, and melanoma. More specifically, glioma cells tested included: D54, U251, U373, and G26; breast tumor cells: 2LMP, DY3672, LCC6, BT474, SK-BR-3, MCF-7, MDA-MB-231, MDA-MB-468, and MDA-MB-453; non-small cell lung carcinoma: A427, WI-62, and H1466; melanoma: SKM28; colorectal cancer: SW948; and prostate cancer cells: PC3, LNCaP, and DU145.

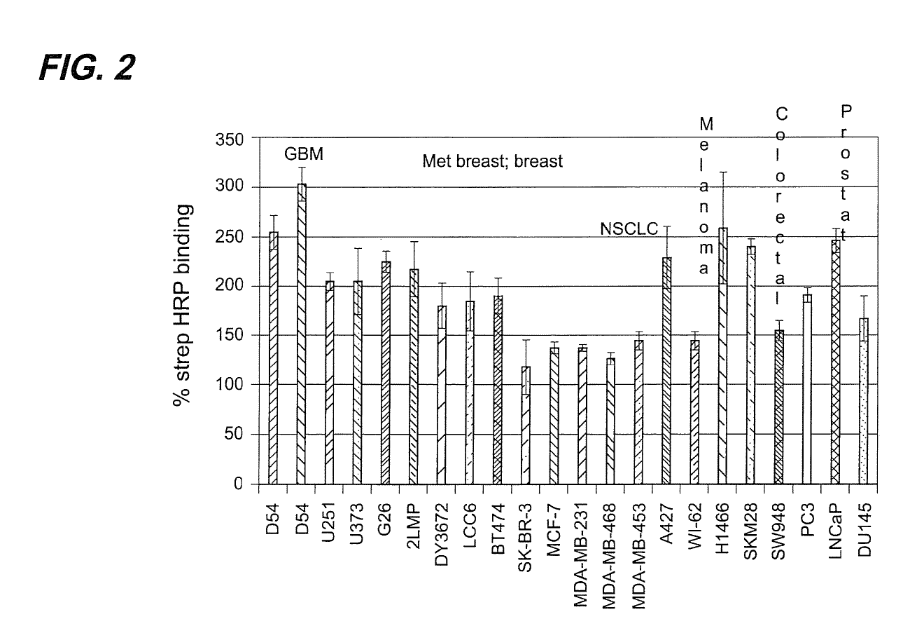

Subconfluent cultures in 96-well plates were assayed for binding by adding TM-602 to live cells followed by addition of streptavidin-HRP to develop the color substrate. Binding was determined as a percent streptavidin-HRP control relative to cells in which no TM-602 was added. All tumor cell lines tested were found to bind TM-602, although the binding to three human breast adenocarcinoma cell lines was less than the other tumor cells tested (see FIG. 2).

Fluorescent activated cell sorting (FACS) was used to demonstrate the reactivity of TM-601 with hematological cancers. Biotinylated TM-601 (TM-602) was used to stain human non-Hodgkin's lymphoma, T-cell leukemia, and myeloma tumor cell lines. By FACS analysis, all hematological tumor cell lines bound to TM-602 (two lymphoma, one T-cell leukemia, and one myelocytic leukemia).

### Example 2

### Non-labeled TM-601 Does Not Decrease Tumor Cell Growth In Vitro

An in vitro cell line screen containing 56 different human tumor cell lines, representing leukemia, melanoma and cancers of the lung, colon, brain, ovary, breast, prostate and kidney was performed by the National Cancer Institute. TM-601 was submitted to the screening service. The results obtained showed that TM-601 was not cytotoxic to any of the cell lines tested. As a follow-up experiment, a single cell line, Panc-1, was tested at a range of serum concentrations (10%, 5%, 2%, 1%) to determine whether cytotoxic activity was evident in low serum growth conditions. No significant cytotoxicity of chlorotoxin was observed, indicating that the primary mechanism of action for chlorotoxin is not via a direct cytotoxic effect on the tumor cells.

### Example 3

### In vitro Tissue Binding of Biotinylated TM-601

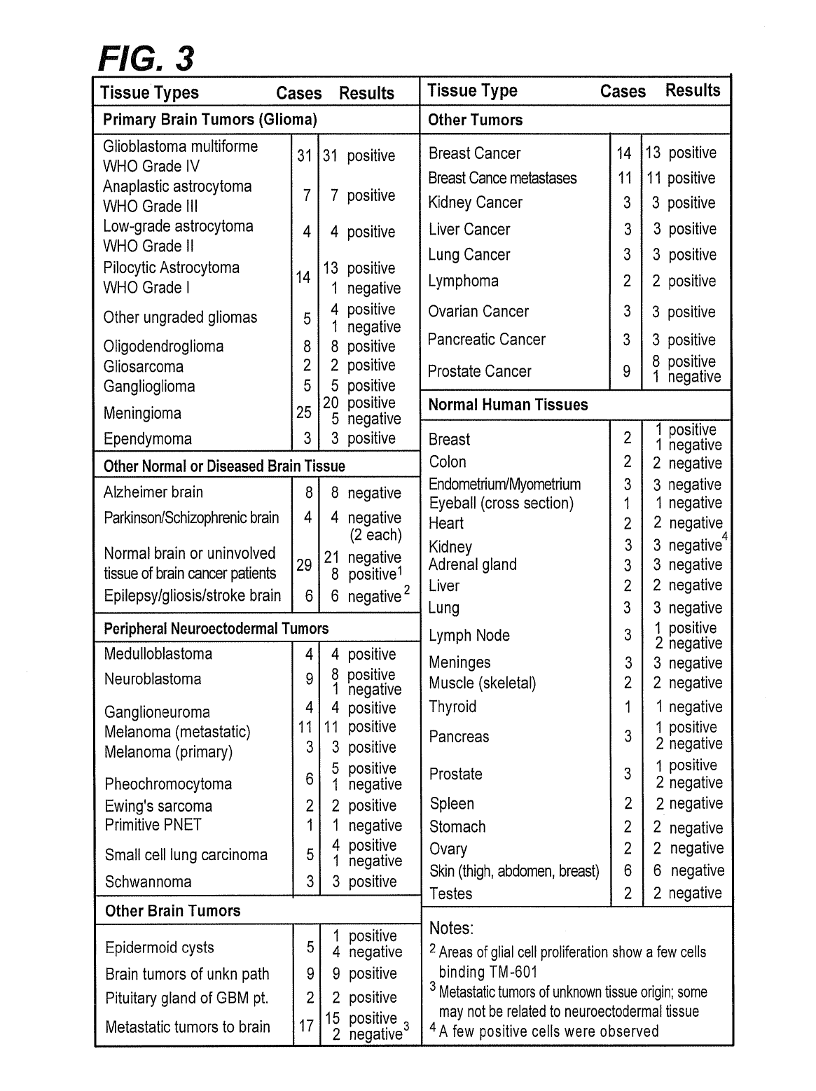

Histochemical staining studies using biotinylated TM-601 (TM-602) were performed to localize TM-601 binding sites on fixed tissues embedded in paraffin or frozen sections of human biopsy and/or autopsy specimens. Thus far, over 200 brain tumor biopsy and autopsy specimens have been evaluated for TM-601 binding. Included in the studies were gliomas, other malignancies, and non-neoplastic tissues. The results of this study and subsequent studies are presented in FIG. 3.

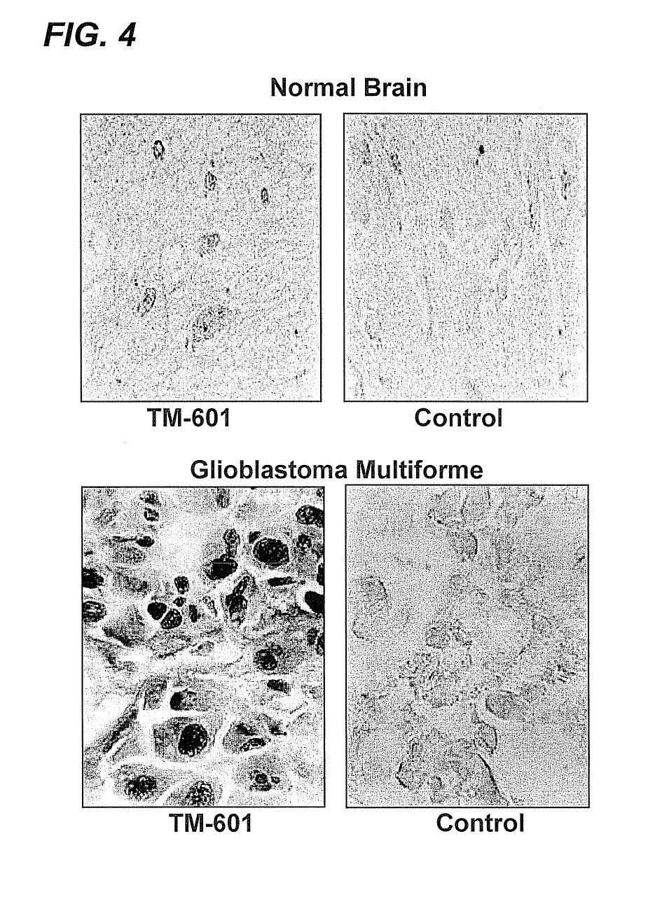

In general, nearly all gliomas demonstrated prominent staining activity for TM-601, while normal, non-tumor tissues did not. The percentage of TM-601 positive cells within a given tumor ranged from 50-98% and increased with increasing WHO malignant grade. An example of glioma specific staining with TM-601 is shown in FIG. 4. In addition, all patient brain tumor tissue samples from a Phase I clinical study showed robust biotinylated TM-601 staining, while non-cancerous brain tissue showed only slight background staining

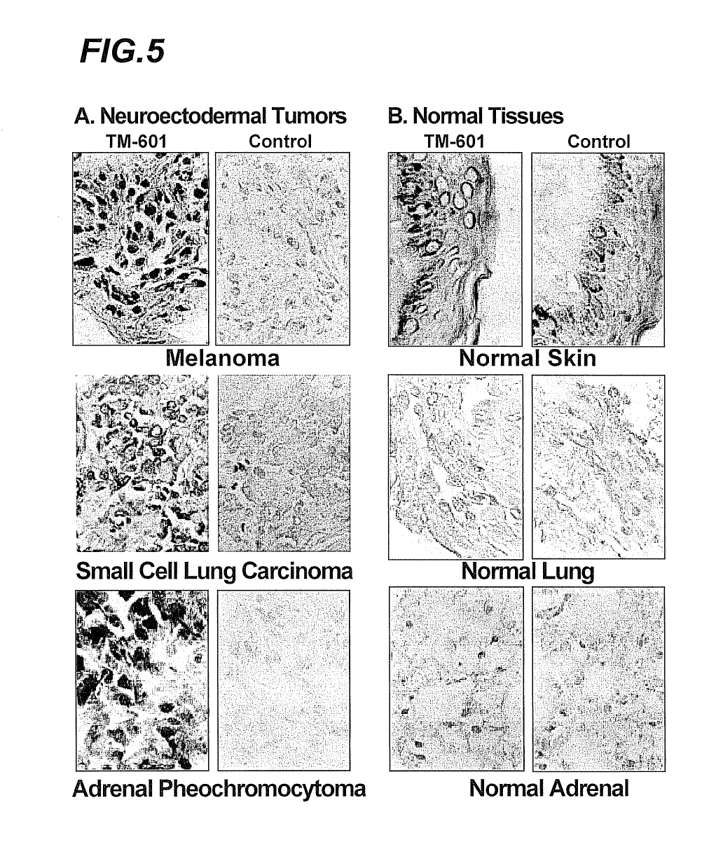

Other positive tissues in this study included peripheral neuroectodermal tumors (PNETs) with an embryonic origin similar to glial tumors. Examples of histochemical staining of PNETs with TM-601 are shown in FIG. 5.

The demonstration of staining in these non-glial tumors suggests that chlorotoxin agents, such as TM-601 could be used to target many tumors other than gliomas. Further studies broadened the types of tumors targeted by TM-601, including breast, breast metastases, prostate, ovary, lung, liver, pancreas, kidney, and lymphoma as examples.

### Example 4

### Efficacy of ¹³¹I-TM-601 in Tumor-Bearing Mice

The ability of ¹³¹I-TM-601 to extend survival in a human glioma xenografted mouse model was studied. Three groups of at least 10 nude mice each were implanted intracranially with the human glioma tumor cell line U251-MG. The U251-MG cells were pre-treated with either saline, non-labeled (“cold”) TM-601 or 1.65 mCi ¹³¹I-TM-601 for 30 minutes ex vivo. The cells were then washed with saline and approximately 1×10⁶ cells were introduced into the brains. The amount of bound radioactivity on the cells receiving ¹³¹I-TM-601 was only 0.2% of the total dose applied to the cells ex vivo. This corresponds to a human brain dose of 0.5 mCi.

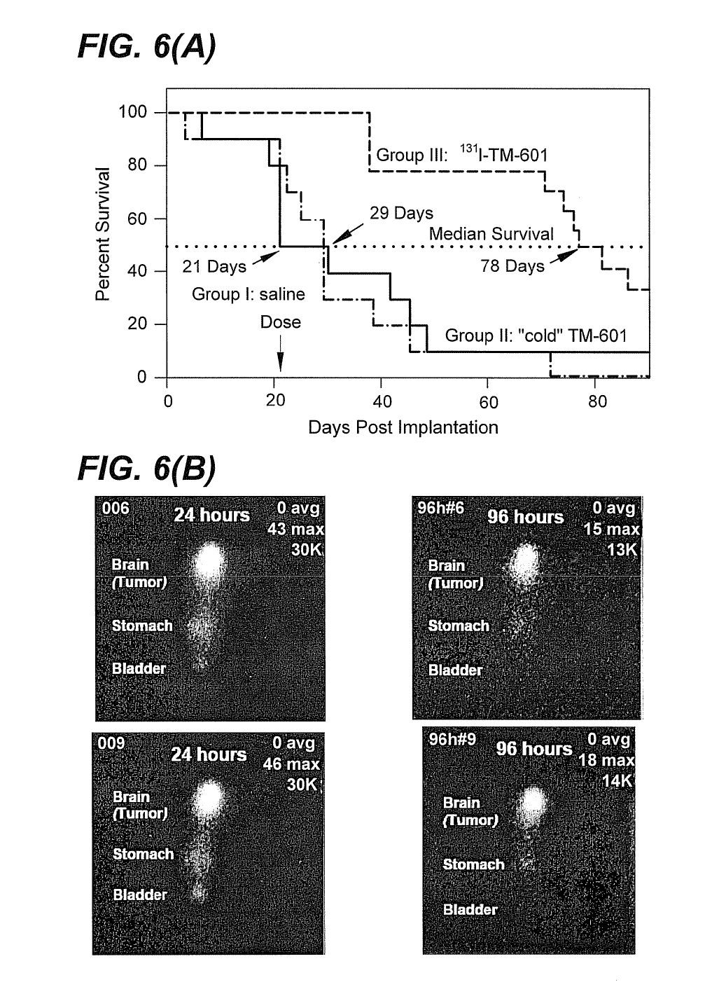

Between 21 and 29 days, the median survival had been reached in the non-labeled TM-601 and saline treated animals, respectively. In contrast, no animals had died in the 131I-TM-601-treated animals during this same period (see FIG. 6(A)). On Day 21, the remaining animals in each group were injected intracranially with their respective ex vivo compound (either saline, non-labeled TM-601, or 0.165 mCi ¹³¹I-TM-601) and gamma camera imaging was performed on Day 22 (24 hours post-treatment) and Day 25 (96 hours post-treatment) on some of the ¹³¹I-TM-601-treated animals. Gamma camera imaging demonstrated the concentration of ¹³¹I-TM-601 in the brain, indicating that the radioactivity had been retained for 24 hours at the site of injection. The imaging further demonstrated that radioactivity when linked to TM-601 was specifically retained in the region of the tumor for more than 96 hours with little uptake by other tissues over this period (see FIG. 6(B)).

Median survival of the ¹³¹I-TM-601-treated group was reached on Day 78 post-tumor implantation, and when the study was terminated at Day 90, five of the 14 ¹³¹I-TM-601-treated animals were still alive. There was a 169% of survival in the group of animals treated with ¹³¹I-TM-601. The drug was well-tolerated by the treated mice with no compound-associated behavior effects noted. These results demonstrate the therapeutic efficacy and diagnostic potential of labeled chlorotoxin agents such as ¹³¹I-TM-601 in a nude mouse model. This is remarkable considering the radiation resistance of the U251-MG tumor cell line and the administration of only two doses of radiation, and further supports the clinical application of chlorotoxin agents for treating and imaging high-grade gliomas.

### Example 5

### ¹²⁵I-TM-601 Intravenously Administered to Tumor-Bearing Mice

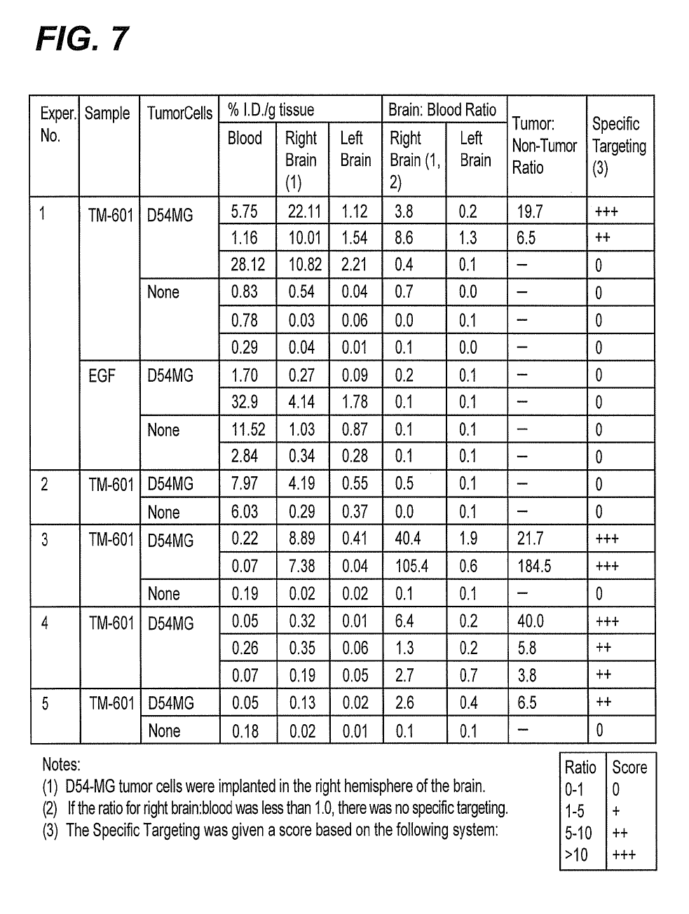

A series of experiments were performed to evaluate the ability of ¹²⁵I-TM-601 to cross the blood brain barrier. These experiments consisted of five research studies utilizing the E54-MG/SCID mouse model. In these studies, ¹²⁵I-TM-601 was injected via the tail vein, and the subsequent ability of TM-601 to target the brain tumor was measured after 24 hours. One experiment also utilized ¹²⁵I-EGF as a control, since it is known that the EGF receptor is up-regulated in these tumors. The use of EGF as a control was important, since EGF, like TM-601, has been shown to bind glioma cells when injected intracranially. As seen in FIG. 7, intravenous injection of ¹²⁵I-TM-601 specifically targeted the D54-MG xenografted human glioma tumors implanted in the right hemisphere of the brains of these animals, demonstrating that ¹²⁵I-TM-601 administered intravenously crosses the blood brain barrier. Also, it is important to note that intravenous injection of ¹²⁵I-EGF did not localize to the brain tumors to any appreciable extent, indicating that it did not cross the intact blood brain barrier. It is concluded from the experiments that chlorotoxin agents such as ¹²⁵I-TM-601 cross the blood brain barrier and reach tumors located in the brain in their biologically active state.

### Example 6

### Efficacy of TM-601 Intravenously Administered to Tumor-Bearing Mice

Given that systemically administered ¹²⁵I-TM-601 can cross the blood brain barrier and bind to intracranial tumors, an experiment was conducted with non-labeled TM-601 to test for anti-tumor activity in the brain using an intravenous route of administration.

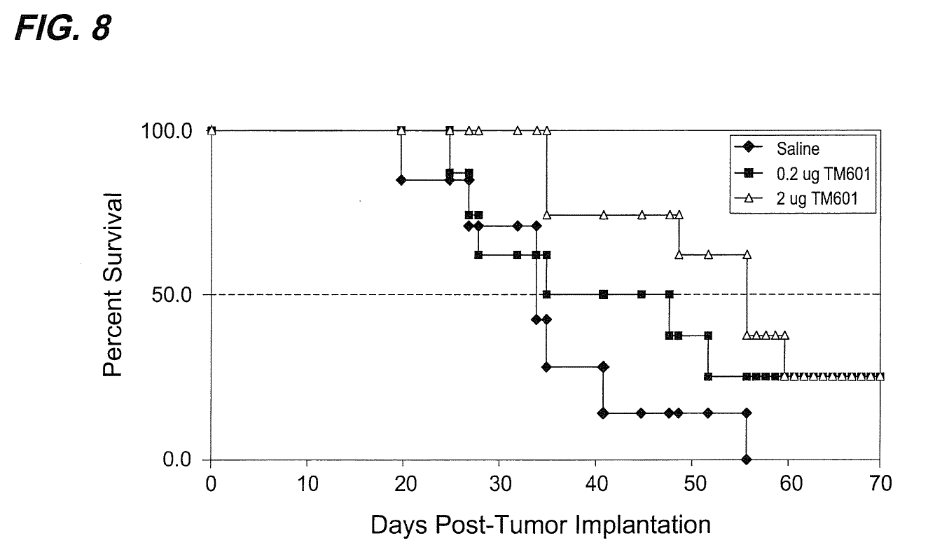

Nude mice were implanted intracranially with D54MG glioma cells. Twice weekly chronic tail vein injection of either saline or two different doses of TM-601 (0. 2 μg or 2.0 μg per mouse per injection) were given beginning 14 days post-implantation of the xenografts for the duration of the study. The survival curves from this experiment demonstrate that enhanced survival occurred in animals given the higher dose of TM-601 (FIG. 8). Measuring the median survival (time when 50% of the animals were alive) high dose TM-601 treatment extended the median survival time from 34 days (saline group) to 56 days in a dose-dependent manner. A lower TM-601 dose did not show the same extension of life. Again, this suggests that chronic systemic dosing of the non-labeled molecule is effective given in a mouse model. Further histological analysis of treated animals with D54MG brain tumors will be necessary to distinguish whether survival prolongation is due to direct effects on tumor cells, or relate to another effect.

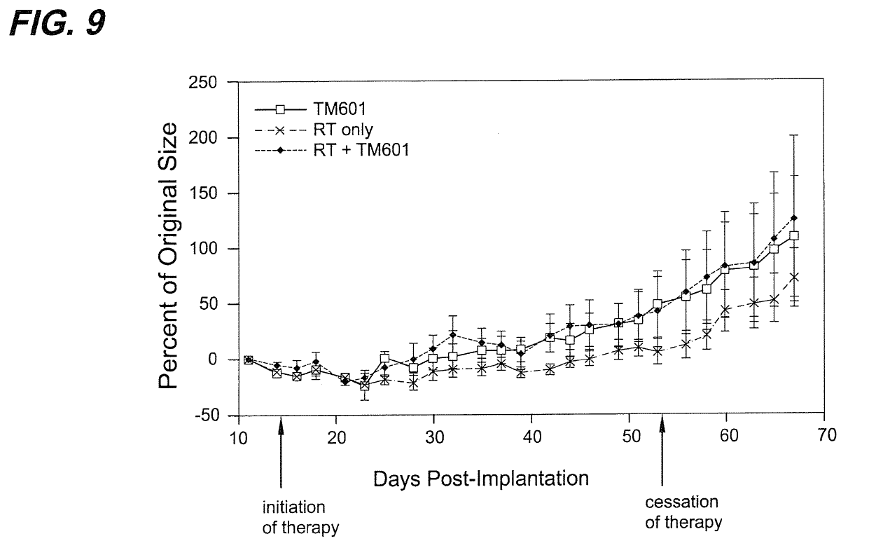

One additional preliminary study which supports the therapeutic potential of non-labeled TM-601 as a monotherapy is a chronic systemic (i.v.) delivery of TM-601 using a mouse flank tumor model. Using this xenogeneic tumor model, D54MG human glioma cells were implanted in the flanks of nude mice (7-8 animals per group). Beginning 14 days later, non-labeled TM-601 was administered twice via tail vein at a dose of 0.26 μg per injection per mouse. One group of animals received TM-601 treatment, one group received 2 Gy of radiation (RT) twice weekly, and the third group received RT and TM-601. Between days 52 and 53, the last dose of TM-601 and/or RT was given, and animals were kept on study until day 67. The tumor growth curves for the TM-601 and the combination TM-601 and RT groups are very closely aligned during therapy and following cessation of therapy (FIG. 9).

The curve for RT group suggests that the tumors grew slower in the RT only group. However, error bars indicating the standard error of the mean demonstrate the level of variability particularly at later time points. Because of this and the small sampling size (at the end of the study n=5 for RT only and n=8 for other groups), the difference in tumor growth did not appear to be statistically significant. This data suggests that both TM-601 and RT prevented disease progression over the course of 38-39 days of therapy and that tumors commenced growing upon completion treatment. To be more conclusive, a control group should have been included to establish the rate of tumor progression in the absence of therapy, however, historically tumors in this D54MG flank model grow faster than observed in this experiment. This preliminary study thus suggests that chronic systemic delivery of TM-601 as a monotherapy may be effective in this flank tumor model.

In summary, in vitro studies with chlorotoxin demonstrated specific binding to a number of tumor cell types, including glioma, neuroectodermal tissue, as well as many other tumors such as breast and prostate. This histological binding was confirmed with tumor cell lines. The binding appears to be selective to tumors as binding to normal tissues was significantly weaker. Using in vivo models, both intracranial injection of ¹³¹I-TM-601 and repeated systemic injections of non-labeled TM-601 have been shown to extend the life of animals with intracranial glioma xenografts. These data suggest that radiolabeled TM-601 can impact tumor growth via localized delivery of a radioactive tag whereas non-labeled TM-601 can impact tumor growth through a yet undefined mechanism.

### Example 7

### Pharmacokinetics and Metabolism of TM-601 in Animals

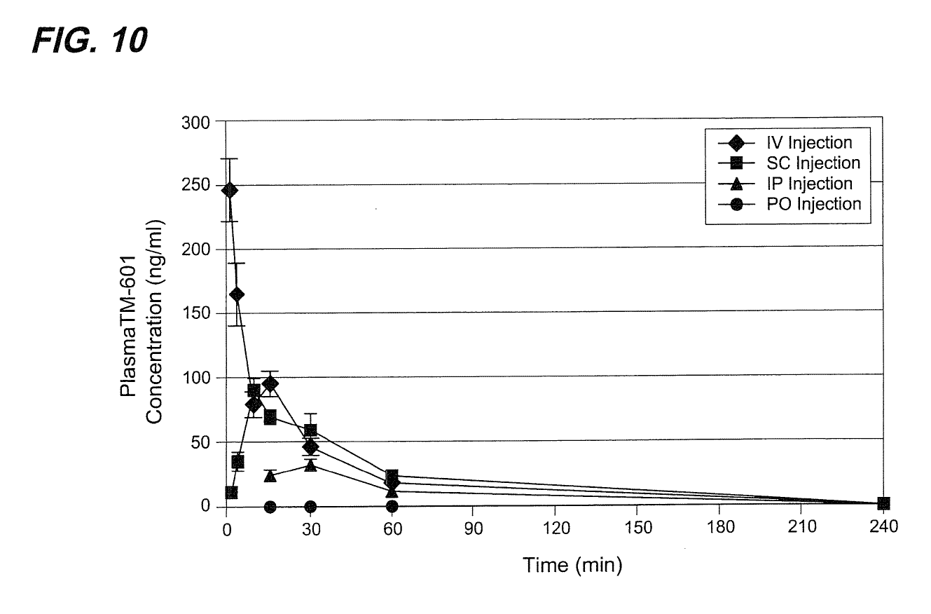

Pharmacokinetics of TM-601 in Nude Mice: Plasma and urine levels of TM-601 were measured following intravenous (IV), intraperitoneal (IP), subcutaneous (SC) or oral gavage (PO) delivery of a single dose of 2 μg TM-601 to nude mice. TM-601 was detected using an ELISA assay that uses a rabbit anti-TM-701 antibody that cross-reacts with TM-601. TM-701 differs from TM-601 by one amino acid (tyrosine substitution at residue 29). Plasma levels following a single dose of 2 μg TM-601 are shown in FIG. 10. The highest peak serum level (Cmax) was observed following intravenous injections, followed by subcutaneous administration, intraperitoneal administration and oral gavage. Plasma levels of TM-601 were not quantifiable when administered via oral gavage.

The half-life of TM-601 in plasma was calculated to be 17.7 minutes for intravenous administration and 27.4 minutes for subcutaneous administration. TM-601 was measured in the urine of animals sacrifices at 30, 60 or 240 minutes after a single 2 μg intraperitoneal or subcutaneous injection. TM-601 is greatly concentrated during renal clearance. Without more information about the total urine production over time, the total amount of TM-601 excreted through the urine and the kinetics of excretion cannot be determined. Within 4 hours from drug administration, when the circulating TM-601 has decreased below the level of assay detection, the concentration in the urine has also dropped below the level of detection. The pharmacokinetics of TM-601 when administered via IV, IP, SC or oral gavage can be roughly classified into three different profiles. Intravenous injections result in a large bolus peak of drug in the blood at the earliest time point measured. The drug then declines with a half-life of approximately 18 minutes. In contrast, either intraperitoneal or subcutaneous delivery resulted in a slower kinetic profile presumable as TM-601 more slowly enters the blood compartment from the site of injection. In addition, the half-life is increased as well to approximately 27 minutes. This increase in half-live could be explained by a more complex blood pharmacokinetic profile in which circulating levels represent a balance between continued release of drug from the subcutaneous injection site and excretion/metabolism. The third type of pharmacokinetic profile was exhibited following oral gavage. Plasma levels were only slightly above the background of the assay and could be quantitated. Thus, with the current formulation, an oral route of administration does not effectively reach systemic circulation.

Taken together, the in vitro and in vivo data obtained suggest that TM-601 binds tumors with high specificity and sensitivity, and that TM-601 can cross the blood brain barrier. In animals, administration of radiolabeled TM-601 was well tolerated and showed high selectivity and excellent retention in tumors.

### Example 8

### Toxicity of TM-601 Intravenously Administered to Animals

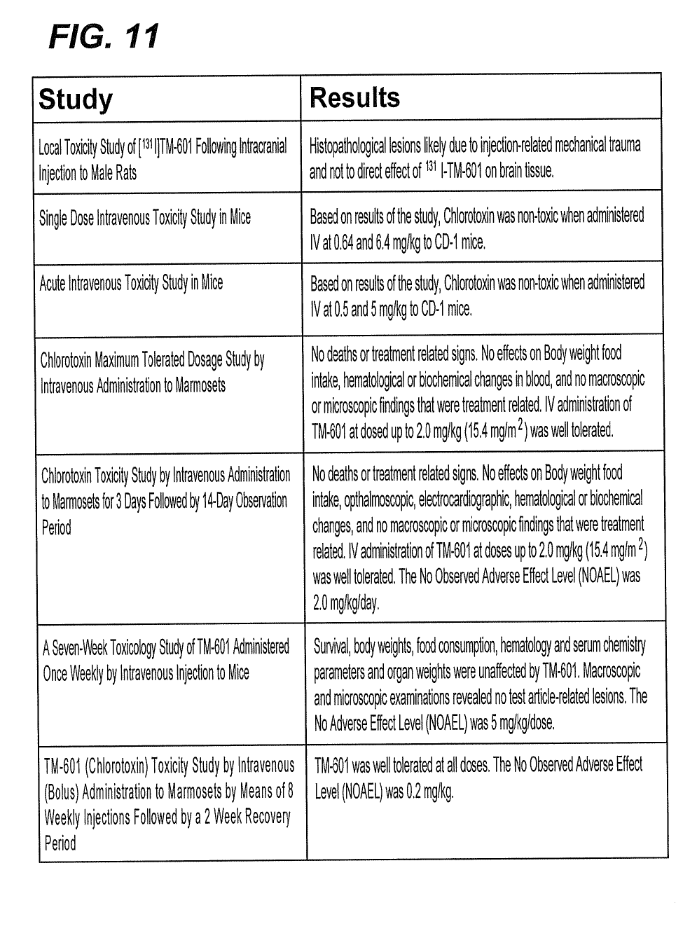

The toxicologic effect of TM-601 has been evaluated in rodents and primates in seven GLP toxicology studies, as summarized in FIG. 11. In six of these 7 studies, TM-601 was administered intravenously. Because no signs of systemic toxicity were observed in any of the 7 studies, the systemic NOAEL (i.e., No-Observed-Adverse-Effect-Level) in each study is equal to or greater than the maximum dose administered.

Single Dose Intravenous Toxicity Study in Mice: A study was conducted in CD-1 mice following IV administration at 0 (vehicle, 0.9% sodium chloride), 0.64 or 6.4 mg/kg (HED of 0.05 and 0.5 mg/kg). TM-601 was reconstituted in sterile saline (0.9% sodium chloride) for injection and administered to 10 mice/sex/group as a single IV dose (10 mL/kg). Evaluations for compound-related effects were based on clinical observations, body weight, food consumption, ophthalmology, and hematology and clinical chemistry parameters. On Day 15 of the study, all surviving animals were sacrificed and subjected to a gross postmortem examination including organ weights and gross and microscopic postmortem examinations. There were no unscheduled deaths during this study. There were no TM-601-related effects on body weight, food consumption or on hematology and clinical chemistry parameters. There were no compound-related changes observed upon ophthalmologic examination. At necropsy, no gross or microscopic lesions attributable to TM-601 administration were observed, nor were there any changes in organ weights.

Acute Intravenous Toxicity Study in Mice: The acute toxicity of TM-601 was assessed in CD-1 mice following intravenous (IV) administration at 0 (vehicle, 0.9% sodium chloride), 0.5 or 5.0 mg/kg. The active compound was approximately 82% of the compound weight. TM-601 was reconstituted in sterile saline (0.9% sodium chloride) for injection and administered to 5 mice/sex/group as a single IV dose (10 mL/kg). Evaluations for compound-related effects were based on clinical observations and body weight. On Day 15, all surviving animals were killed and subjected to a gross postmortem examination. Dose solutions were analyzed to verify the accuracy of the dose. From the 0.5 mg/kg group, one female and one male were found dead on Day 6 and another male was cold to the touch and had decreased activity on this day. It appeared that these animals had insufficient access to drinking water that resulted in dehydration. No animals died at the 5.0 mg/kg dosage. There were no TM-601-related effects on body weights and no visible lesions were observed in any animals that died during the study or at terminal necropsy.

Based on the findings from the single-dose intravenous toxicology studies in mice, TM-601 is considered to be non-toxic after single IV doses of 0.64 and 6.4 mg/kg. The IV NOAEL dose is therefore at least 6.4 mg/kg in the mouse (HED of 0.5 mg/kg).

Repeat Dose Toxicity

Chlorotoxin Maximum Tolerated Dosage Study by Intravenous Administration to Marmosets: The acute toxicity of TM-601 was assessed in marmoset monkeys (Callithryx jacchus) in a pyramiding study design. In this range finder study, one male and one female marmoset were administered TM-601 IV at 0.020, 0.20, and 2.0 mg/kg (HED of 0.003, 0.03 and 0.3 mg/kg, respectively) with a 3-day wash-out period between each dose. TM-601 was dissolved in 0.9% sodium chloride and administered intravenously in volumes of 0.4, 0.4 and 2.0 mL/kg body weight for the 3 dosages, respectively. Evaluations for compound related effects were based on clinical observations, body weights, food consumption and hematology, coagulation, and clinical chemistry parameters. One day after administration of the last dose, animals were euthanized, organ weights were taken, and gross and microscopic pathology were performed on any observed tissue abnormalities and injection sites.

There were no unscheduled deaths during this study nor were there any TM-601-related findings in any of the parameters examined.

Chlorotoxin Toxicity Study by Intravenous Administration to Marmosets for 3 Days Followed by 14-Day Observation Periods: The toxicity of TM-601 was assessed in marmoset monkeys following 3 consecutive days of IV administration. Marmosets (3/sex/dose) were administered TM-601 at 0 (0.9% sterile saline), 0.20 or 2.0 mg/kg/day (HED of 0.03 and 0.3 mg/kg, respectively). Evaluations for compound-related effects were based on clinical observations, body weights, food consumption and hematology, coagulation, and clinical chemistry parameters. Animals were observed for 14 days after the last (third) dose administration before being euthanized, and organ weights were taken. Gross and microscopic pathology were performed on all tissues.

There were no unscheduled deaths in this study. There were no compound-related effects on clinical observations, body weights, food consumption and hematology, coagulation, and clinical chemistry parameters. Additionally, there were no TM-601-related effects on organ weights, no signs of compound effects at necropsy, and no histopathological findings attributed to compound.

The no-effect dose of TM-601 administered intravenously to marmosets for 3 consecutive days is at least 2.0 mg/kg (HED of 0.3 mg/kg).

A Seven-Week Toxicology Study of TM-601 Administered Once Weekly by Intravenous Injection to Mice: The chronic toxicity of systemically administered TM-601, when delivered by intravenous injection, was performed in mice. Eight total doses, administered by bolus injection in the tail vein once weekly for 7 weeks, were given to three groups of Crl:CD-1® (ICR)BR mice in a dose volume of 5 mL/kg. Dosage levels were 0.5, 2, and 5 mg/kg/dose. A concurrent control group received the vehicle on a comparable regimen. Following 7 weeks of dose administration, 10 mice/sex/group were euthanized; the remaining 3-5 mice/sex in the control and high dose groups were euthanized following a 14-day non-dosing (recovery) period. Animals were observed twice daily for mortality and morbidity, and clinical examinations were performed daily. Clinical pathology evaluations (hematology and serum chemistry) were performed just prior to the primary (study week 7) and recovery (study week 9) necropsies. Complete necropsies were conducted on all animals, and selected organs were weighed at the scheduled necropsies.

The results of these studies indicated that survival, body weights, food consumption, hematology and serum chemistry parameters and organ weights were unaffected by administration of TM-601. Macroscopic and microscopic examinations revealed no test article-related lesions. Possible test article-related clinical findings consisted of ptosis and hypoactivity at low incidences in the 2 and 5 mg/kg/dose group at 1 hour following dosing. In all cases, these clinical findings were not observed on non-dosing days and were not considered adverse.

Based on the results of this study, the NOAEL for weekly intravenous administration of TM-601 to mice for 7 weeks (8 total doses) was at least 5 mg/kg/dose (HED of 0.4 mg/kg).

TM-601 (Chlorotoxin) Toxicity Study by Intravenous (Bolus) Administration to Marmosets by Means of 8 Weekly Injections Followed by a 2 Week Recovery Period: The systemic chronic toxic potential of TM-601 to common marmosets (Callithrix jaccus) when administered once weekly by intravenous injection was assessed over a period of 7 weeks. Dosage levels were 0.06 or 0.2 mg/kg. Following 7 weeks of dose administration, 3 animals/sex/group were euthanized; the remaining 2 animals/sex/group in the control and high dose groups were euthanized following a 14-day nondosing (recovery) period. A control group received the vehicle (phosphate buffered saline) at the same frequency. Clinical conditions, bodyweight, food consumption, toxicokinetics, urinalysis, organ weight, macropathology and histopathology investigations were undertaken.

Signs seen at the dose administration site consisted predominantly of bruising with occasional thickening and scabbing, and the incidence included control and treated animals alike, and is considered to be associated with the route of administration and not the result of treatment of TM-601.

Bodyweight change was variable, and showed the periodic fluctuations typical of marmosets. Again, there was no consistent difference between control and treatment groups, no difference within treatment groups, and there was no statistical significance seen in this parameter. Food consumption was similar in treated and control groups.

Clinical pathology indicated no difference between the treated and the control groups. Urinalysis similarly showed no differences. Organ weights did not show any treatment related inter-group differences. Microscopic examination indicated that there were no treatment effects in any organs evaluated.

The conclusion of this study was that the administration of 8 intravenous injections of TM-601 at dosages of 0.06 and 0.20mg/kg was very well tolerated with no evidence of any adverse effect of treatment or systemic toxicity. Signs seen at the sites of administration at the in life observations and at the macroscopic and microscopic examinations were associated with low level physical trauma caused by the route of administration and were not a consequence of exposure to chlorotoxin. The NOAEL as identified by this study was considered to be at least 0.2 mg/kg (HED of 0.03 mg/kg).

### Example 9

### Phase I Imaging and Safety Study of Intravenous ¹³¹I-TM-601 in Patients with Recurrent or Refractory Somatic and/or Cerebral Metastatic Solid Tumors

A Phase I imaging trial has been completed at 5 clinical sites, administering TM-601 intravenously to a total of 48 patients. This multi-center, open-label, non-randomized, sequential “within subject” escalation study included patients with histologically confirmed primary solid tumor malignancy, either recurrent or refractory, that had demonstrated unequivocal evidence of detectable metastatic involvement that was not amenable to standard therapy.

Objectives of this Phase I study were: a) to evaluate whether intravenous ¹³¹I-TM-601 provides tumor-specific localization in patients with recurrent or refractory metastatic (including brain metastases) solid tumors; b) to determine the distribution and dosimetry of intravenously administered ¹³¹I-TM-601; and c) to determine the safety and tolerability of intravenously administered ¹³¹I-TM-601.

Patients and Treatment Protocol

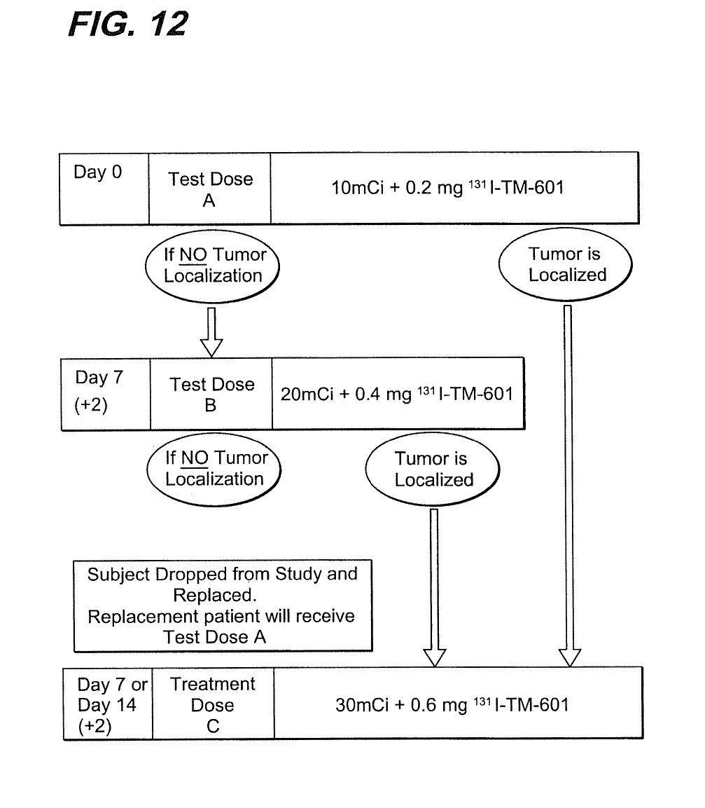

Approximately 50 subjects were enrolled in this study, undergoing 2 to 3 escalating intravenous doses of ¹³¹I-TM-601 followed by a series of whole body scans to determine whether the ¹³¹I-TM-601 had localized to target tumor cells, and one intravenous therapeutic dose of TM-601 once tumor-specific uptake of ¹³¹I-TM-601 had been demonstrated. The graphic in FIG. 12 illustrates the dosing scheme.

Study patients received up to three escalating doses of ¹³¹I-TM-601 (ranging from 10 mCi/0.2 mg to 30 mCi/0.6 mg) by intravenous (IV) infusion. Only patients demonstrating tumor specific uptake of ¹³¹I-TM-601 by imaging performed 24 hours after administration of the 10 or 20 mCi dose received treatment with the 30 mCi dose of ¹³¹I-TM601.

Preparation of ¹³¹I-TM-601

The final TM-601 drug product is a sterile, lyophilized white to off-white powder vialed in stoppered glass vials. Imaging and therapeutic doses used in this trial were doses of radiolabeled TM-601.

Preparation and Use of¹³¹I-TM-601: TM-601 final drug product was reconstituted in 0.56 mL of radio-labelling buffer to yield a 1 mg/mL solution radio-labeled with ¹³¹I, and delivered to the clinical site. The syringe contained approximately 4 mL of solution for infusion and was approximately labeled as to content and amount of radioactivity. Once received at the site, the radiation safety officer or other appropriate site personnel confirmed that radiation count of the ¹³¹I-TM-601 was within prescribed specifications. The syringe containing the final radio-labeled drug product was shielded and then transferred to the appropriate hospital area for administration to the patient. The ¹³¹I-TM-601 solution was stored protected from light at 2-8° C. and shielded until use. After radio-labelling with ¹³¹I, the product was used within 24 hours.

### Administration of ¹³¹I-TM-601 and Imaging Study

All patients receiving the radio-labeled test dose, ¹³¹I-TM-601, received supersaturated potassium iodide (SSKI), at a dose of 300 mg/day orally, beginning on the day of and just prior to radio-labeled ¹³¹I-TM-601 infusion and for a minimum of three days to block uptake of ¹³¹I to the thyroid and other organs. SSKI was dispensed to the patient prior to study drug administration with instructions provided to the patient on the proper use of the drug while not in the clinic/hospital.

The syringe containing the ¹³¹I-TM-601 was inserted “piggy-back” fashion into an infusion port within six-inches of the intravenous needle/catheter. While running 0.9% sodium chloride at 100 mL/hour, the product was administered by “slow IV push” over approximately 5-10 minutes. ¹³¹I-TM-601 infusion was terminated if any of the following conditions arose: (1) a fall in systolic blood pressure >25 mmHg, (2) a significant respiratory distress documented by the investigator, (3) temperature >102° F., (4) seizures, (5) changes in level of consciousness or onset of new neurological deficit, or other reasons, such as clinician's judgment or patient's request.

Imaging by gamma camera and in some cases SPECT was performed 24 hours post ¹³¹I-TM-601 administration to determine localization and eligibility for receiving the 30 mCi dose of ¹³¹I-TM-601.

### Safety Results

As of April 2007, 17 patients had received at least two doses of the IV-administered treatment (up to 30 mCi/0.6 mg) with no acute adverse experiences. Seven unique patients experienced a total of 7 Serious Adverse Effects (SAEs). None of the SAEs were assessed by the Investigator as “possibly” or “probably related” to study drug. One patient died within 30 days of dosing. This patient was a 60 year old man with a history of metastatic small cell lung cancer who enrolled in the study and received two IV doses of ¹³¹I-TM-601 on Oct. 25, 2006 and Nov. 1, 2006. The patient did not have any acute reactions to therapy, nor any tumor specific uptake, and subsequently went on to receive palliative radiation to the spine. The patient was enrolled in hospice and expired at home on Nov. 24, 2006. The investigator assessed the patient's death as “unlikely related” to study agent and more likely related to progressive disease. Two patients experienced one SAE each prior to dosing (bilateral pulmonary emboli and UTI, CTCAE Grade 4 and 3, respectively); one patient experienced abdominal pain and distention one day after dosing (CTCAE Grade 4); one patient was unable to walk 10 day after dosing and was found to have spinal cord compression (CTCAE Grade 3) presumed secondary to progression of underlying disease; and 2 patients experienced a DVT 18-20 days after dosing (both CTCAE Grade 3).

### Efficacy Results

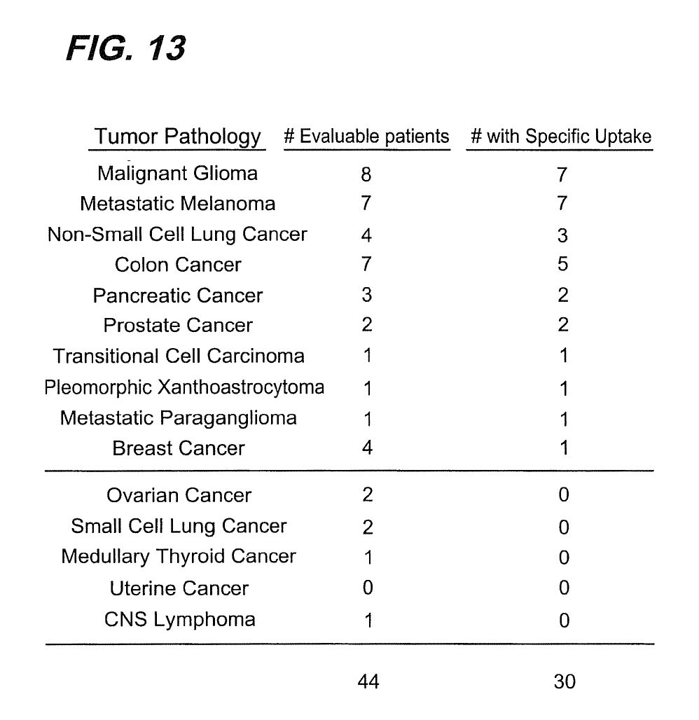

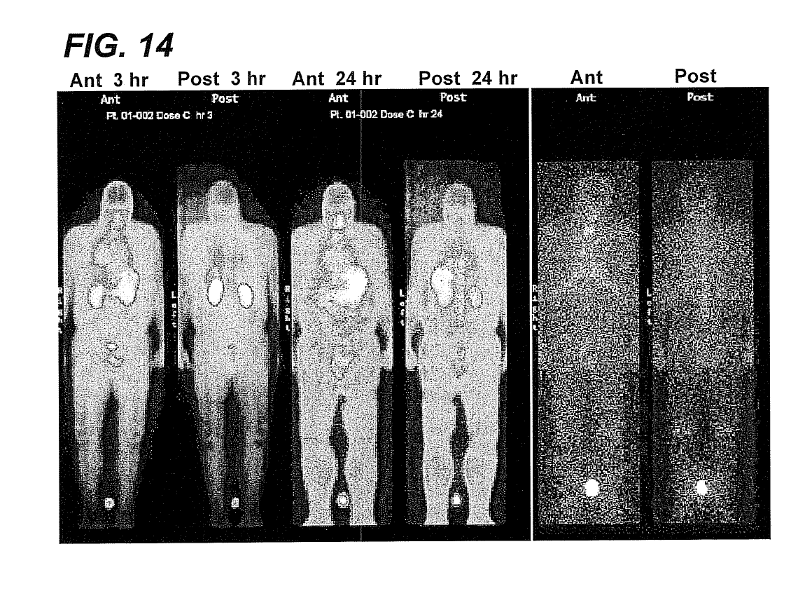

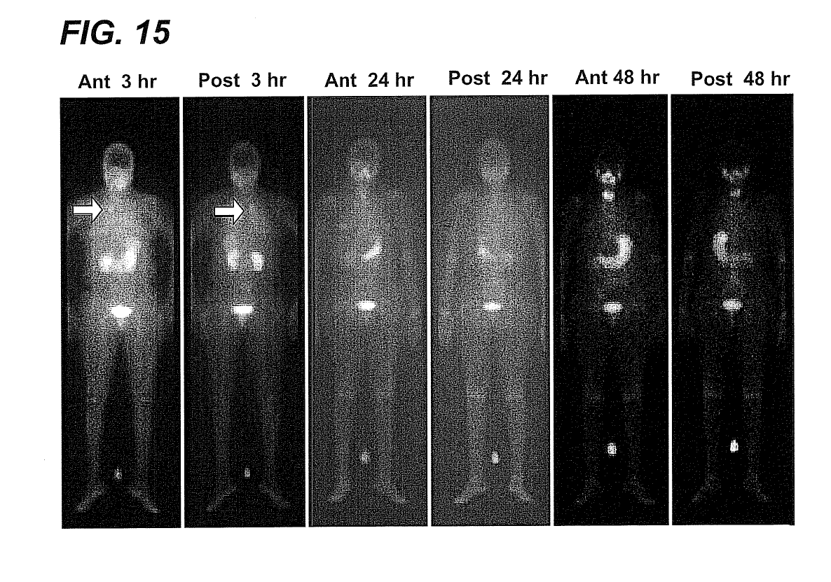

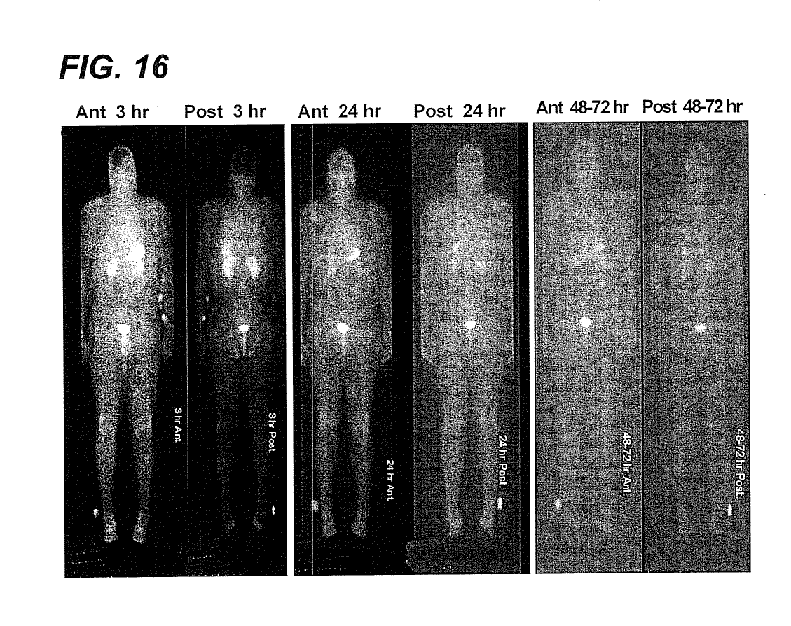

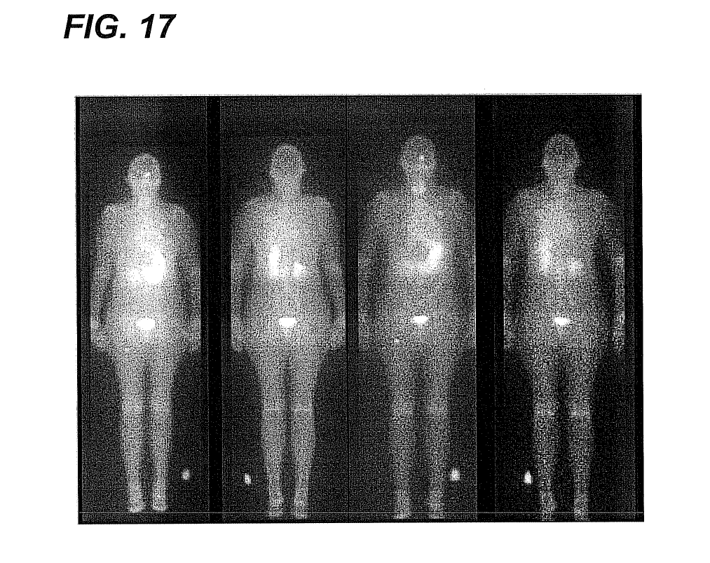

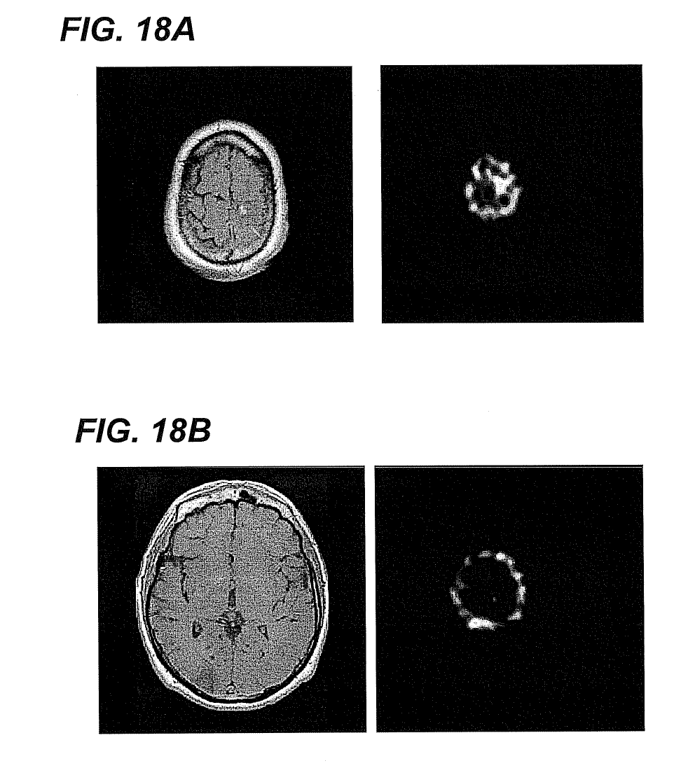

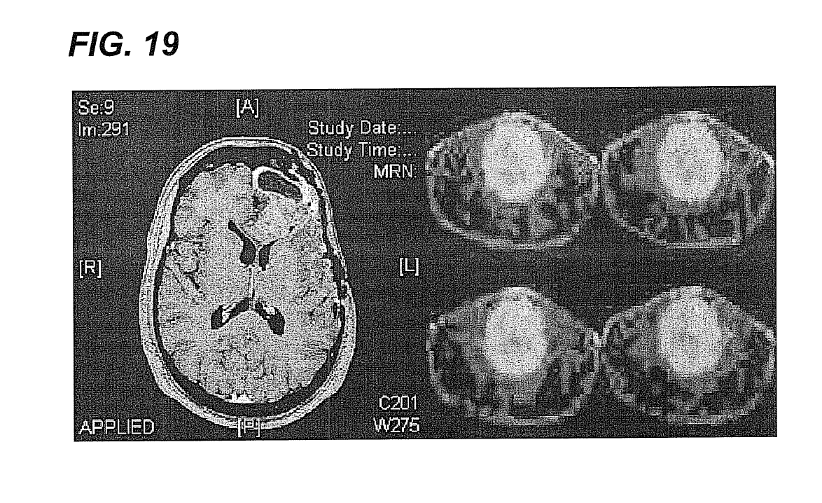

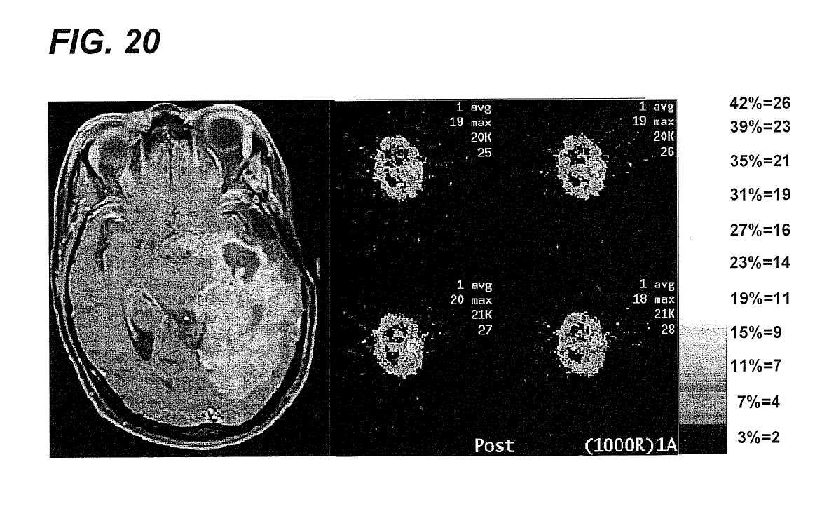

Tumor specific uptake was seen in a variety of tumor types following intravenous administration, including 7 out of 8 patients with malignant glioma, 7 out of 7 patients with metastatic melanoma, 2 of 2 patients with prostate cancer, 3 of 4 patients with non-small cell lung cancer, and 5 of 7 patients with metastatic colon cancer (as summarized on FIG. 13, see also FIGS. 14-20).

All patients received a test dose of 10 mCi (0.2mg peptide) ¹³¹I-TM-601 intravenously. Five sequential, whole body gamma camera images were acquired at immediate, 3 hours, 24 hours, 48-72 hours, and 168 hours post ¹³¹I-TM-601 injection for tumor localization and dosimetry analysis. Patients showing tumor localization by gamma camera or SPECT imaging received a second therapeutic dose of 30 mCi (0.6mg peptide) ¹³¹I-TM-601 one week later. Patients not showing uptake were re-treated a week later with 20 mCi (0.4mg peptide) 131I-TM-601 to determine possible localization at a higher dose.

All seven patients with glioma included in a dosimetry subset analysis demonstrated tumor specific localization on follow-up gamma camera or SPECT imaging after IV administration of ¹³¹I-TM-601. Dose limiting toxicity was not observed. The mean radiation dose was 0.23 cGy/mCi (ranged 0.15-0.31 cGy/mCi) to the whole body and 0.81 cGy/mCi (ranged 0.36-1.51 cGy/mCi) to tumor with a calculated therapeutic ratio of approximately 3.5 (tumor/body).

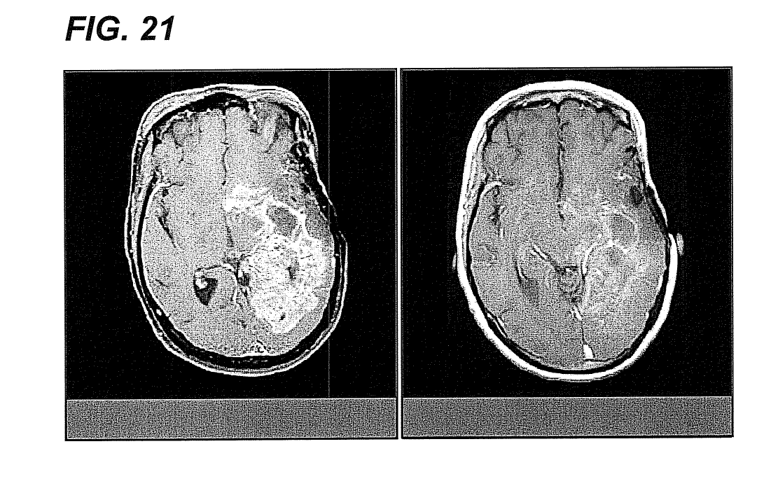

On preliminary imaging analysis, one patient with malignant glioma demonstrated a significant reduction in enhancing tumor volume and edema 3 weeks following treatment (see FIG. 21). MRI imaging on this patient at the day 21 evaluation demonstrated a reduction in the T1 enhancing volume and T2 volume. Another patient with malignant glioma, who also showed tumor-specific uptake of ¹³¹I-TM-601 and who then received the intravenous treatment dose, exhibited apparent clinical improvement in the absence of imaging improvement.

These results demonstrate the therapeutic effect of chlorotoxin agents such as TM-601 delivered systemically in vivo. These results also demonstrate that ¹³¹I-TM-601 administered intravenously will cross the blood brain barrier and can result in MR imaging improvement in patients with inoperable gliomas.

### Example 10

### Increasing and/or Altering Therapeutic Effect of Chlorotoxin Agents

The present Example demonstrates that increasing the total amount of a chlorotoxin agent that a subject is exposed to (i.e., increasing the area under the curve observed after dosing) can increase and/or alter thetherapeutic effect of the agent.

As described in International Patent Application serial number PCT/US08/76740 filed Sep. 17, 2008, pegylation of a chlorotoxin agent can increase its half life in blood and furthermore can increase its ability to inhibit angiogenesis.

Without wishing to be bound by any particular theory, the present invention proposes that such observations made with PEGylated chlorotoxin agents may represent an area under the curve effect. For example, it is well known that different therapeutic agents trigger biological effects in different ways. Some require achievement of a particular threshold level, for example within a particular amount of time; some require a level of total exposure; some have a combination of such requirements. The present invention proposes that some effects of chlorotoxin agents (e.g., specific binding, possibly cellular uptake) may be achieved at low doses or exposure levels, but that higher total exposure (e.g., area under the curve) may be required to inhibit angiogenesis and/or to achieve other therapeutic effects.

### Materials and Methods

### PEGylation

TM-601 was PEGylated at the N-terminus of the peptide via reductive amination using a polydispersed, linear, 40 kDa PEG-propionaldehyde (DowPharma).

### Half-Life Measurements of TM-601

Non-tumor-bearing C57BL/6 mice were injected with TM-601 (at a dose of approximately 2 mg/kg) intravenously by a single tail vein injection. Blood samples were obtained at various timepoints, and levels of TM-601 were determined by ELISA using an anti-TM-601 antibody.

### Mouse Matrigel Plug

Matrigel Matrix High Concentration (from BD Biosciences) was mixed with 100 ng/ml VEGF, 100 ng/ml bFGF, and 3 ng/ml heparin at 4° C. Eight-week old female C57BL/6 mice were randomly assigned to each groups with 6 mice in each group. Each mouse received two 500 μL Matrigel plugs injected bilaterally in subcutaneous tissue. To form a round shaped plug, a wide subcutaneous pocket was formed by swaying the needlepoint right and left after a routine subcutaneous insertion. The injection was performed rapidly with a 21-25G needle to ensure the entire contents were delivered into the plug. Matrigel plugs were implanted on Day 0 of the study and treatment began on Day 1. Animals were dosed with intravenous injections with either vehicle (saline), TM-601, or PEGylated TM-601. Three dosing regimens were used: once a week for two weeks (once on D1 and once on D8; “Q7Dx2”), twice a week for two weeks (on D1, D4, D8, and D11; “Q3Dx2/2”), and five times a week for two weeks (on D1, D2, D3, D4, D5, D8, D9, D10, D11, and D12; “Q1Dx5/2”). Plugs were collected after 14 days. Mice were euthanized and the skin over the plugs was pulled back. Plugs were dissected out, fixed, and embedded in paraffin for histological analysis. Three sections of 5 μm thickness from each evaluable plug were immunostained with a CD31 antibody and counterstained with hematoxylin & eosin. Blood vessel counts in a cross sectional area of each matrigel plug was analyzed under a microscope.

### Results/Discussion

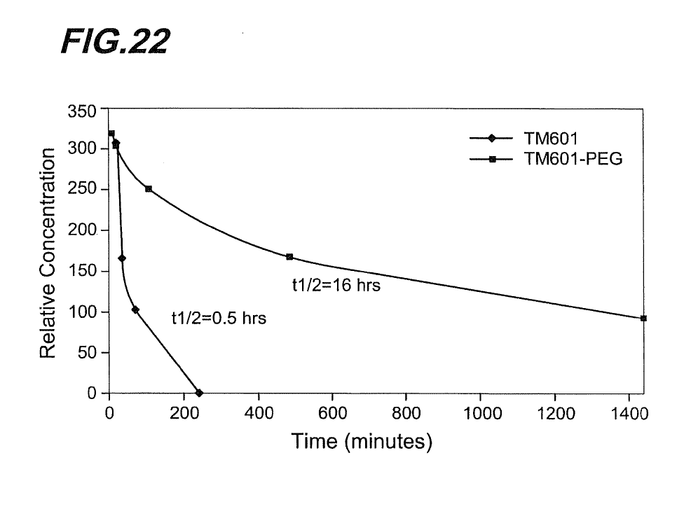

As shown in FIG. 22, PEGylated TM-601 exhibited an increased half-life in vivo as compared to unmodified TM-601. Surprisingly, PEGylation increased the half life of TM-601 approximate 32-fold, that is, approximately 25 minutes (TM-601) to approximately 16 hrs (TM-601-PEG).

Increased half life translated into the ability to dose the animals less frequently in a model of angiogenesis. In mouse Matrigel plug assays, animals were dosed according to a variety of schedules with either TM-601 or PEGylated TM-601 (TM-601-PEG). Microvessel densities were measured and reduction of such densities was interpreted to signify anti-angiogenic effects.

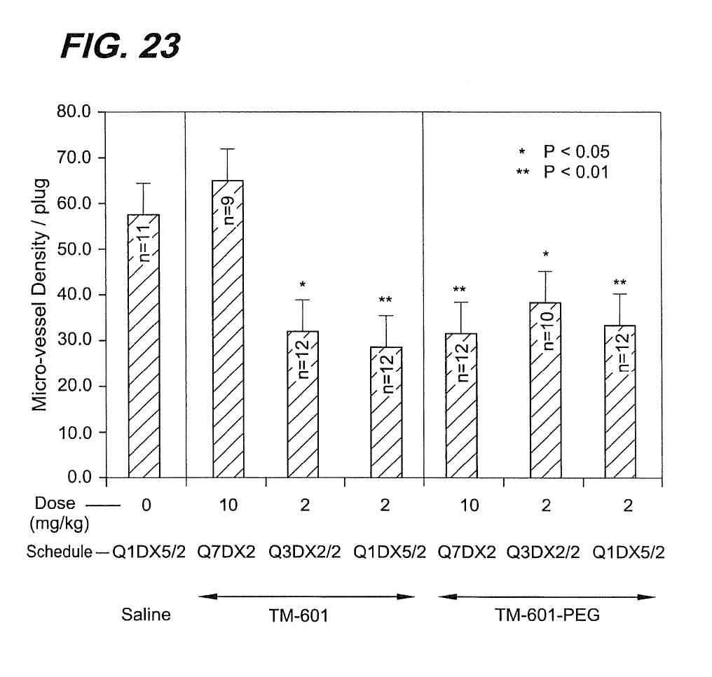

Both TM-601 and TM-601-PEG had anti-angiogenic effects with the two most frequent dosing schedules tested (twice a week for two weeks, “Q3DX2/2”; and five times a week for two weeks , “Q1Dx5/2”) (FIG. 23). Whereas TM-601 did not exhibit any anti-angiogenic effects with the least frequent dosing schedule tested (once a week for two weeks, “Q7DX2”) treatment with TM-601-PEG with such a dosing schedule resulted in a significant reduction of micro-vessel density (FIG. 23).

Without wishing to be bound by any particular theory, the ability to dose animals less frequently may be due to availability of TM-601-PEG for a longer period of time as compared to TM-601. Such increased availability could result in longer exposure at sites of new blood vessel formation, allowing more prolonged therapeutic effect. Further without wishing to be bound by any particular theory, it is possible that such longer exposure (i.e., increased area under the curve) in fact permits a therapeutic effect that would not otherwise be observed, even for example, under conditions that permit binding and/or uptake of TM-601 (or another chlorotoxin agent).

The improved therapeutic effects observed with TM-601-PEG as compared with TM-601 are surprising, among other things, because chlorotoxin is a relatively small peptide, so that one would expect significant modification such as PEGylation would be likely to alter or compromise function. The data presented herein surprisingly demonstrate that a PEGylated chlorotoxin agent not only retains activity (e.g., binding activity) but in fact shows enhanced and/or new activity (e.g., anti-angiogenic effects).

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method of treating an individual with a tumor, the method comprising a step of: administering an effective amount of a chlorotoxin agent to the individual, wherein the chlorotoxin agent is administered systemically.

2. The method of claim 1, wherein the chlorotoxin agent is administered intravenously.

3. The method of claim 1, wherein the chlorotoxin agent selectively targets cancer cells over normal cells.

4. The method of claim 1, wherein the chlorotoxin agent comprises a chlorotoxin moiety selected from the group consisting of chlorotoxin, biologically active chlorotoxin subunits, and chlorotoxin derivatives.

5. The method of claim 1, wherein the chlorotoxin agent comprises a chlorotoxin moiety associated with at least one therapeutic moiety.

6. The method of claim 5, wherein the chlorotoxin moiety and therapeutic moiety are directly covalently associated.

7. The method of claim 5, wherein the chlorotoxin moiety and therapeutic moiety are fused to form a fusion protein.

8. The method of claim 5, wherein the chlorotoxin moiety and therapeutic moiety are covalently associated through a linker.

9. The method of claim 5, wherein the therapeutic moiety comprises an anti-cancer agent.

10. The method of claim 9, wherein the anti-cancer agent is selected from the group consisting of alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens.

11. The method of claim 5, wherein the therapeutic moiety comprises a cytotoxic agent.

12. The method of claim 11, wherein the cytotoxic agent is selected from the group consisting of toxins, bioactive proteins, chemotherapeutic antibiotics, nucleolytic enzymes, and radioisotopes.

13. The method of claim 12, wherein the cytotoxic agent comprises a member of the group consisting of gelonin, ricin, saponin, Pseudomonas exotoxin, pokeweed antiviral protein, diphtheria toxin, and complement proteins.

14. The method of claim 11, wherein the cytotoxic agent comprises a radioisotope.

15. The method of claim 14, wherein the cytotoxic agent comprises iodine-131 (¹³¹I).

16. The method of claim 1, wherein the tumor is a solid tumor.

17. The method of claim 1, wherein the tumor is a refractory tumor.

18. The method of claim 1, wherein the tumor is a recurrent tumor.

19. The method of claim 1, wherein the tumor is a metastatic tumor.

20. The method of claim 1, wherein the tumor is a member of the group consisting of lung cancer, bone cancer, liver cancer, pancreatic cancer, skin cancer, squamous cell cancer, non-small cell lung carcinoma, cancer of the head or neck, cutaneous or intraocular melanoma, malignant melanoma, uterine cancer, ovarian cancer, prostate cancer, rectal cancer, cancer of the anal region, stomach cancer, colon cancer, breast cancer, uterine cancer, carcinoma of the sexual or reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemia, lymphocytic lymphomas, cancer of the bladder, cancer of the kidney, renal cell carcinoma, neoplasms of the central nervous system (CNS), neuroectodermal cancer, spinal axis tumors, glioma, meningioma, and pituitary adenoma.

21. The method of claim 1, wherein the tumor is a tumor of neuroectodermal origin.

22. The method of claim 21, wherein the tumor of neuroectodermal origin is a member of the group consisting of glioma, glioblastoma multiforme, meningioma, ependymoma, medulloblastoma, neuroblastoma, ganglioma, pheochromocytoma, melanoma, peripheral primitive neuroectodermal tumor, small cell carcinoma of the lung, Ewing's sarcoma, and metastatic tumor of neuroectodermal origin in the brain.

23. The method of claim 1, wherein the step of administering comprises systemic administration of at least one dose of chlorotoxin agent.

24. The method of claim 23, wherein the dose of chlorotoxin agent comprises between approximately 0.001 mg/kg and approximately 5 mg/kg.

25. The method of claim 1 further comprising a step of detecting the tumor prior to administering the chlorotoxin agent to the individual.

26. The method of claim 25, wherein detecting the tumor comprises steps of:
administering to the individual an effective amount of a labeled chlorotoxin agent, wherein the labeled chlorotoxin agent is administered systemically; and
measuring binding of the labeled chlorotoxin agent to tissue, wherein an elevated level of binding, relative to normal tissue, indicates that the tissue is tumor tissue.

27. The method of claim 26, wherein the labeled chlorotoxin agent is administered intravenously.

28. The method of claim 26, wherein the labeled chlorotoxin agent is labeled with at least one labeling moiety selected from the group consisting of fluorophores, radioisotopes, and paramagnetic metal ions.

29. The method of claim 28, wherein the labeling moiety comprises iodine-131 (¹³¹I) or iodine-125 (¹²⁵I).

30. The method of claim 28, wherein the labeling moiety comprises technetium-99m (^{99m}Tc).

31. The method of claim 28, wherein the labeling moiety comprises copper-64 (⁶⁴Cu).

32. The method of claim 26, wherein the step of measuring binding of the labeled chlorotoxin agent to tissue is performed using a technique selected from the group consisting of Laser-Induced Fluorescence Spectroscopy, Gamma camera, Single Photon Emission Computed Tomography (SPECT), and Positron Emission Tomography (PET).

33. The method of claim 1 further comprising administering a chemotherapeutic agent to the individual.

34. The method of claim 33, wherein the chemotherapeutic agent is selected from the group consisting of alkylating agents, purine antagonists, pyrimidine antagonists, plant alkaloids, intercalating antibiotics, aromatase inhibitors, anti-metabolites, mitotic inhibitors, growth factor inhibitor, cell cycle inhibitors, enzymes, topoisomerase inhibitors, biological response modifiers, anti-hormones and anti-androgens.

35. A method of detecting tumor tissue in an individual, the method comprising steps of:
administering to the individual an effective amount of a labeled chlorotoxin agent, wherein the labeled chlorotoxin agent is administered systemically; and
measuring binding of the labeled chlorotoxin agent to a tissue of interest, wherein an elevated level of binding, relative to normal tissue, indicates that the tissue of interest is tumor tissue.

36. The method of claim 35, wherein the labeled chlorotoxin agent is administered intravenously.

37. The method of claim 35, wherein the labeled chlorotoxin agent selectively targets cancer cells over normal cells.

38. The method of claim 35, wherein the labeled chlorotoxin agent comprises a chlorotoxin moiety selected from the group consisting of chlorotoxin, biologically active chlorotoxin subunits, and chlorotoxin derivatives.

39. The method of claim 35, wherein the labeled chlorotoxin agent is labeled with at least one labeling moiety, wherein the labeling moiety is selected from the group consisting of fluorophores, radioisotopes, and paramagnetic metal ions.

40. The method of claim 35, wherein the labeling moiety comprises iodine-131 (¹³¹I) or iodine-125 (¹²⁵I).

41. The method of claim 35, wherein the labeling moiety comprises technetium-99m (^{99m}Tc).

42. The method of claim 35, wherein the labeling moiety comprises copper-64 (⁶⁴Cu).

43. The method of claim 39, wherein the step of measuring binding of the labeled chlorotoxin agent to tissue is performed using a technique selected from the group consisting of Laser-Induced Fluorescence Spectroscopy, Gamma camera, Single Photon Emission Computed Tomography (SPECT), and Positron Emission Tomography (PET).

44. The method of claim 35, wherein the tumor tissue is from a solid tumor.

45. The method of claim 35, wherein the tumor tissue is from refractory tumor.

46. The method of claim 35, wherein the tumor tissue is from a recurrent tumor.

47. The method of claim 35, wherein the tumor tissue is from a metastatic tumor.

48. The method of claim 35, wherein the tumor tissue is from a tumor selected from the group consisting of lung cancer, bone cancer, liver cancer, pancreatic cancer, skin cancer, squamous cell cancer, non-small cell lung carcinoma, cancer of the head or neck, cutaneous or intraocular melanoma, malignant melanoma, uterine cancer, ovarian cancer, prostate cancer, rectal cancer, cancer of the anal region, stomach cancer, colon cancer, breast cancer, uterine cancer, carcinoma of the sexual or reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, lymphocytic lymphomas, cancer of the bladder, cancer of the kidney, renal cell carcinoma, neoplasms of the central nervous system (CNS), neuroectodermal cancer, spinal axis tumors, glioma, meningioma, and pituitary adenoma.

49. The method of claim 35, wherein the tumor tissue is from a tumor of neuroectodermal origin.

50. The method of claim 49, wherein the tumor of neuroectodermal origin is a member of the group consisting of glioma, glioblastoma multiforme, meningioma, ependymoma, medulloblastoma, neuroblastoma, ganglioma, pheochromocytoma, melanoma, peripheral primitive neuroectodermal tumor, small cell carcinoma of the lung, Ewing's sarcoma, and metastatic tumor of neuroectodermal origin in the brain.

51. The method of claim 35, wherein the step of administering comprises systemic administration of at least one dose of labeled chlorotoxin agent.

52. The method of claim 51, wherein the step of administering comprising systemic administration of a first and second doses of labeled chlorotoxin agent, wherein the second dose is higher than the first dose.

53. The method of claim 51, wherein the step of administering comprises systemic administration of a first, second and third doses of labeled chlorotoxin agent, wherein the second dose is higher than the first dose and the third dose is higher than the second dose.

54. The method of claim 4 or 35, wherein the chlorotoxin moiety is a chlorotoxin derivative has an amino acid sequence comprising a fragment at least 10 amino acids long that shows at least 90% identity to a fragment of SEQ ID NO:1.
